(19) United States
(12) Patent Application Publication
(10) Pub. No.: US 2003/0088274 A1
(43) Pub. Date: May 8, 2003

(54) Method and apparatus for electrically stimulating cells implanted in the nervous system

(75) Inventors: Bradford Evan Gliner, Sammamish, WA (US); Alan J. Levy, Bellevue, WA (US); Jeffrey Blazer, Allison Park, PA (US); Andrew D. Firlik, Ridgefield, CT (US); W. Douglas Sheffield, Loveland, OH (US)

Correspondence Address: PERKINS COIE LLP, PATENT-SEA, P.O. BOX 1247, SEATTLE, WA, 98111-1247, US

(73) Assignee: Vertis Neuroscience, Inc.

(21) Appl. No.: 10/261,116

(22) Filed: Sep. 30, 2002

Related U.S. Application Data
(60) Provisional application No. 60/325,830, filed on Sep. 28, 2001.

(51) Int. Cl. A61N 1/18

(52) U.S. Cl. 607/3

(57) ABSTRACT

The following disclosure describes several methods and apparatus for stimulating cells implanted in the regions of nervous system, such as the brain, spinal cord or peripheral nerves. Accordingly, the functionality of the cells can be improved, for example, by differentiating undifferentiated or partially undifferentiated cells into neurons or other cells having action potentials. The method can also include promoting directional growth and connectivity of fully differentiated neural cells implanted in a patient's nervous system through electrical enhancement, for example, electrical stimulation via an anode and cathode. Methods in accordance with the invention can be used to treat brain damage (e.g., stroke, trauma, etc.), brain disease (e.g., Alzheimer's, Pick's, Parkinson's, etc.), and/or brain disorders (e.g., epilepsy, depression, etc.). The methods in accordance with the invention can also be used to enhance neural-function of normal, healthy brains (e.g., learning, memory, etc.), or to control sensory functions (e.g., pain).

### CROSS-REFERENCE TO RELATED APPLICATIONS

This application claims the benefit of U.S. application Ser. No. 09/802,808, filed Mar. 8, 2001, which claims the benefit of U.S. Provisional Application No. 60/217,981, filed Jul. 31, 2000, both of which are incorporated herein in their entireties by reference. This application also claims the benefit of U.S. Provisional Application No. 60/325,830, filed Sep. 28, 2001 and incorporated herein in its entirety by reference.

### TECHNICAL FIELD

Several embodiments of methods and apparatus in accordance with the invention are related to electrically stimulating cells before and/or after being implanted in the nervous system of a patient to enhance the ability of cells to achieve increased functionality.

### BACKGROUND

A wide variety of mental and physical processes are known to be controlled or are influenced by neural activity in particular regions of the brain.

In some areas of the brain, such as in the sensory or motor cortices, the organization of the brain resembles a map of the human body; this is referred to as the “somatotopic organization of the brain.” There are several other areas of the brain that appear to have distinct functions that are located in specific regions of the brain in most individuals. For example, areas of the occipital lobes relate to vision, regions of the left inferior frontal lobes relate to language in the majority of people, and regions of the cerebral cortex appear to be consistently involved with conscious awareness, memory, and intellect. This type of location-specific functional organization of the brain, in which discrete locations of the brain are statistically likely to control particular mental or physical functions in normal individuals, is herein referred to as the “functional organization of the brain.”

Many problems or abnormalities with body functions can be caused by damage, disease and/or disorders of the nervous system, which includes the brain, the spinal cord and the peripheral nerves. A stroke, for example, is one very common condition that damages the brain. Strokes are generally caused by emboli (e.g., obstruction of a vessel), hemorrhages (e.g., rupture of a vessel), or thrombi (e.g., clotting) in the vascular system of a specific region of the cortex, which in turn generally causes a loss or impairment of a neural function (e.g., neural functions related to face muscles, limbs, speech, etc.). Stroke patients are typically treated using physical therapy to rehabilitate the loss of function of a limb or another affected body part. For most patients, little can be done to improve the function of the affected limb beyond the recovery that occurs naturally without intervention. One existing physical therapy technique for treating stroke patients constrains or restrains the use of a working body part of the patient to force the patient to use the affected body part. For example, the loss of use of a limb is treated by restraining the other limb. Although this type of physical therapy has shown some experimental efficacy, it is expensive, time-consuming and little-used. Stroke patients can also be treated using physical therapy plus adjunctive therapies. For example, some types of drugs, such as amphetamines, that increase the activation of neurons in general, appear to enhance neural networks; these drugs, however, have limited efficacy because they are very non-selective in their mechanisms of action and cannot be delivered in high concentrations directly at the site where they are needed. Therefore, there is a need to develop effective treatments for rehabilitating stroke patients and patients that have other types of brain damage.

Other brain disorders and diseases are also difficult to treat. Alzheimer's disease, for example, is known to affect portions of the cortex, but the cause of Alzheimer's disease and how it alters the neural activity in the cortex is not fully understood. Similarly, the neural activity of brain disorders (e.g., depression and obsessive-compulsive behavior) is also not fully understood. Therefore, there is also a need to develop more effective treatments for other brain disorders and diseases.

The neural activity in the brain can be influenced by electrical energy that is supplied from an external source outside of the body. Various neural functions can thus be promoted or disrupted by applying an electrical current to the cortex or other region of the brain. As a result, the quest for treating damage, disease and disorders in the brain have led to research directed toward using electricity or magnetism to control brain functions.

One type of treatment is transcranial electrical stimulation (TES), which involves placing an electrode on the exterior of the scalp and delivering an electrical current to the brain through the scalp and skull. Patents directed to TES include: U.S. Pat. No. 5,540,736 issued to Haimovich et al. (for providing analgesia); U.S. Pat. No. 4,140,133 issued to Katrubin et al. (for providing anesthesia); U.S. Pat. No. 4,646,744 issued to Capel (for treating drug addiction, appetite disorders, stress, insomnia and pain); and U.S. Pat. No. 4,844,075 issued to Liss et al. (for treating pain and motor dysfunction associated with cerebral palsy). TES, however, is not widely used because the patients experience a great amount of pain and the electrical field is difficult to direct or focus accurately.

Another type of treatment is transcranial magnetic stimulation (TMS), which involves producing a high-powered magnetic field adjacent to the exterior of the scalp over an area of the cortex. TMS does not cause the painful side effects of TES. Since 1985, TMS has been used primarily for research purposes in brain-mapping endeavors. Recently, however, potential therapeutic applications have been proposed primarily for the treatment of depression. A small number of clinical trials have found TMS to be effective in treating depression when used to stimulate the left prefrontal cortex.

The TMS treatment of a few other patient groups have been studied with promising results, such as patients with Parkinson's disease and hereditary spinocerebellar degeneration. Patents and published patent applications directed to TMS include: published international patent application WO 98/06342 (describing a transcranial magnetic stimulator and its use in brain mapping studies and in treating depression); U.S. Pat. No. 5,885,976 issued to Sandyk (describing the use of transcranial magnetic stimulation to treat a variety of disorders allegedly related to deficient serotonin neurotransmission and impaired pineal melatonin functions); and U.S. Pat. No. 5,092,835 issued to Schurig et al. (describing the treatment of neurological disorders (such as autism), treatment of learning disabilities, and augmentation of mental and physical abilities of “normal” people by a combination of transcranial magnetic stimulation and peripheral electrical stimulation).

Independent studies have also demonstrated that TMS is able to produce a lasting change in neural activity within the cortex that occurs for a period of time after terminating the TMS treatment (“neuroplasticity”). For example, Ziemann et al., Modulation of Plasticity in Human Motor Cortex after Forearm Ischemic Nerve Block, 18 J Neuroscience 1115 (February 1998), disclose that TMS at subthreshold levels (e.g., levels at which movement was not induced) in neuro-block models that mimic amputation was able to modify the lasting changes in neural activity that normally accompany amputation. Similarly, Pascual-Leone et al. (submitted for publication) disclose that applying TMS over the contralateral motor cortex in normal subjects who underwent immobilization of a hand in a cast for 5 days can prevent the decreased motor cortex excitability normally associated with immobilization. Other researchers have proposed that the ability of TMS to produce desired changes in the cortex may someday be harnessed to enhance neuro-rehabilitation after a brain injury, such as stroke, but there are no published studies to date.

Other publications related to TMS include Cohen et al., Studies of Neuroplasticity With Transcranial Magnetic Stimulation, 15 J. Clin. Neurophysiol. 305 (1998); Pascual-Leone et al., Transcranial Magnetic Stimulation and Neuroplasticity, 37 Neuropsychologia 207 (1999); Stefan et al., Induction of Plasticity in the Human Motor Cortex by Paired Associative Stimulation, 123 Brain 572 (2000); Sievner et al., Lasting Cortical Activation after repetitive TMS of the Motor Cortex, 54 Neurology 956 (February 2000); Pascual-Leone et al., Study and Modulation of Human Cortical Excitability With Transcranial Magnetic Stimulation, 15 J. Clin. Neurophysiol. 333 (1998); and Boylan et al., Magnetoelectric Brain Stimulation in the Assessment Of Brain Physiology And Pathophysiology, 111 Clin. Neurophysiology 504 (2000).

Although TMS appears to be able to produce a change in the underlying cortex beyond the time of actual stimulation, TMS is not presently effective for treating many patients because the existing delivery systems are not practical for applying stimulation over an adequate period of time. TMS systems, for example, are relatively complex and require stimulation treatments to be performed by a healthcare professional in a hospital or physician's office. TMS systems also may not be reliable for longer-term therapies because it is difficult to (a) accurately localize the region of stimulation in a reproducible manner, and (b) hold the device in the correct position over the cranium for a long period, especially when a patient moves or during rehabilitation. Furthermore, current TMS systems generally do not focus the electromagnetic energy on the desired region of the cortex for a sufficient amount of time. As such, the potential therapeutic benefit of TMS using existing equipment is relatively limited.

Direct and indirect electrical stimulation of the central nervous system has also been proposed to treat a variety of disorders and conditions. For example, U.S. Pat. No. 5,938,688 issued to Schiff notes that the phenomenon of neuroplasticity may be harnessed and enhanced to treat cognitive disorders related to brain injuries caused by trauma or stroke. Schiff's implant is designed to increase the level of arousal of a comatose patient by stimulating deep brain centers involved in consciousness. To do this, Schiff's invention involves electrically stimulating at least a portion of the patient's intralaminar nuclei (i.e., the deep brain) using, e.g., an implantable multipolar electrode and either an implantable pulse generator or an external radiofrequency controlled pulse generator. Schiff's deep brain implant is highly invasive, however, and could involve serious complications for the patient.

Likewise, U.S. Pat. No. 6,066,163 issued to John acknowledges the ability of the brain to overcome some of the results of an injury through neuroplasticity. John also cites a series of articles as evidence that direct electrical stimulation of the brain can reverse the effects of a traumatic injury or stroke on the level of consciousness. The system disclosed in John stimulates the patient and modifies the parameters of stimulation based upon the outcome of comparing the patient's present state with a reference state in an effort to optimize the results. Like Schiff, however, the invention disclosed in John is directed to a highly invasive deep brain stimulation system.

Another device for stimulating a region of the brain is disclosed by King in U.S. Pat. No. 5,713,922. King discloses a device for cortical surface stimulation having electrodes mounted on a paddle implanted under the skull of the patient. The electrodes are implanted on the surface of the brain in a fixed position. The electrodes in King accordingly cannot move to accommodate changes in the shape of the brain. King also discloses that the electrical pulses are generated by a pulse generator that is implanted in the patient remotely from the cranium (e.g., subclavicular implantation). The pulse generator is not directly connected to the electrodes, but rather it is electrically coupled to the electrodes by a cable that extends from the remotely implanted pulse generator to the electrodes implanted in the cranium. The cable disclosed in King extends from the paddle, around the skull, and down the neck to the subclavicular location of the pulse generator.

King discloses implanting the electrodes in contact with the surface of the cortex to create paresthesia, which is a sensation of vibration or “buzzing” in a patient. More specifically, King discloses inducing paresthesia in large areas by applying electrical stimulation to a higher element of the central nervous system (e.g., the cortex). As such, King discloses placing the electrodes against particular regions of the brain to induce the desired paresthesia. The purpose of creating paresthesia over a body region is to create a distracting stimulus that effectively reduces perception of pain in the body region. Thus, King appears to require stimulation above activation levels.

Although King discloses a device that stimulates a region on the cortical surface, this device is expected to have several drawbacks. First, it is expensive and time-consuming to implant the pulse generator and the cable in the patient. Second, it appears that the electrodes are held at a fixed elevation that does not compensate for anatomical changes in the shape of the brain relative to the skull, which makes it difficult to accurately apply an electrical stimulation to a desired target site of the cortex in a focused, specific manner.

Third, King discloses directly activating the neurons to cause paresthesia, which is not expected to cause entrainment of the activity in the stimulated population of neurons with other forms of therapy or adaptive behavior, such as physical or occupational therapy. Thus, King is expected to have several drawbacks.

King and the other foregoing references are also expected to have drawbacks in producing the desired neural activity because these references generally apply the therapy to the region of the brain that is responsible for the physiological function or mental process according to the functional organization of the brain. In the case of a brain injury or disease, however, the region of the brain associated with the affected physiological function or cognitive process may not respond to stimulation therapies. Thus, existing techniques may not produce adequate results that last beyond the stimulation period.

Cell replacement therapy is another method for restoring functionality lost to several systems of the body due to damage, disease and or disorders of the central nervous system. Dead or dysfunctional cells in the brain or spinal cord are replaced by undifferentiated cells, such as stem cells or blast cells. These cells may be derived from cultured cells, dedifferentiated cell lines, cancer cell lines, fetal tissues or other progenitor cell types. These relatively undifferentiated cells transform themselves to replace and assume the duties of native cells lost due to disease, damage or trauma. Accordingly, the implanted cells can assume many characteristics of the native cells that they are replacing. One method of cell replacement therapy (disclosed in U.S. Pat. No. 6,214,334 to Lee) is to implant mature neurons at the site of nerve damage. The mature neurons can develop as replacement cells for the destroyed or damaged neurons and can make necessary linkages to restore the functionality of the damaged neurons. However, the process of cell replacement therapy does not always result in full or even partial functionality of the replacement cells.

Another method of cell replacement currently available for promoting recovery from damage to the central nervous system involves implanting stem cells within the brain or spinal cord and administering a neural stimulant to the cells, as described in published international patent application WO01/12236 to Finklestein, et al. Finklestein discloses administering stem cells and a neural stimulant in vivo to improve sensory, motor or cognitive abilities. In preferred embodiments, the neural stimulant is an anti-depressant, such as Prozac, an amphetamine, such as Ridilin, a tricyclic anti-depressant such as Elavil, or combinations thereof. In another embodiment, the neural stimulant may be TMS.

Another type of cell replacement therapy for promoting the growth and proliferation of nerves cells is disclosed in U.S. Pat. No., 6,095,148 to Shastri, et al. Shastri discloses a method for promoting attachment, proliferation, and differentiation of nerve cells by electrical stimulation of the cells on electrically conductive polymers. More specifically, Shastri discloses attaching or abutting nerve cells to an electrically conductive polymer and applying a voltage or current to the polymer. Shastri and the other foregoing references are expected to have drawbacks in achieving full functionality of the replacement cells. Additionally, the replacement cells may not grow in the desired directions to complete functional connections with other cells.

### BRIEF DESCRIPTION OF THE DRAWINGS

FIG. 1A is a schematic view of neurons.

FIG. 1B is a graph illustrating firing an “action potential” associated with normal neural activity.

FIG. 1C is a flowchart of a method for effectuating a neural-function of a patient associated with a location in the brain in accordance with one embodiment of the invention.

FIG. 2 is a top plan view of a portion of a brain illustrating neural activity in a first region of the brain associated with the neural-function of the patient according to the somatotopic organization of the brain.

FIG. 3 is a top plan image of a portion of the brain illustrating a loss of neural activity associated with the neural-function of the patient used in one stage of a method in accordance with an embodiment of the invention.

FIG. 4 is a top plan image of the brain of FIG. 3 showing a change in location of the neural activity associated with the neural-function of the patient at another stage of a method in accordance with an embodiment of the invention.

FIGS. 5A and 5B are schematic illustrations of an implanting procedure at a stage of a method in accordance with an embodiment of the invention.

FIG. 5C is a graph illustrating firing an “action potential” associated with stimulated neural activity in accordance with one embodiment of the invention.

FIG. 5D is a flowchart illustrating a method in accordance with one embodiment of the invention for electrically stimulating cells implanted in a nervous system of a patient.

FIG. 5E is a flowchart of a method in accordance with another embodiment of the invention for electrically stimulating cells implanted in a nervous system of a patient.

FIG. 5F is a flowchart of a method in accordance with still another embodiment of the invention for electrically enhancing cells implanted in a nervous system of a patient.

FIG. 6 is an isometric view of an implantable stimulation apparatus in accordance with one embodiment of the invention.

FIG. 7 is a cross-sectional view schematically illustrating a part of an implantable stimulation apparatus in accordance with an embodiment of the invention.

FIG. 8 is a schematic illustration of a pulse system in accordance with one embodiment of the invention.

FIG. 9 is a schematic illustration of an implanted stimulation apparatus and an external controller in accordance with an embodiment of the invention.

FIG. 10 is a schematic illustration of an implantable stimulation apparatus having a pulse system and an external controller in accordance with another embodiment of the invention.

FIG. 11 is a cross-sectional view schematically illustrating a part of an implantable stimulation apparatus in accordance with an embodiment of the invention.

FIG. 12 is a schematic illustration of an implantable stimulation apparatus having a pulse system and an external controller in accordance with another embodiment of the invention.

FIG. 13 is a cross-sectional view schematically illustrating a part of an implantable stimulation apparatus having a pulse system and an external controller in accordance with another embodiment of the invention.

FIG. 14 is a bottom plan view and

FIG. 15 is a cross-sectional view illustrating an electrode configuration for an implantable stimulation apparatus in accordance with an embodiment of the invention.

FIG. 16 is a bottom plan view and

FIG. 17 is a cross-sectional view of an electrode configuration for an implantable stimulation apparatus in accordance with another embodiment of the invention.

FIG. 18 is a bottom plan view and

FIG. 19 is a cross-sectional view of an electrode configuration in accordance with yet another embodiment of the invention.

FIG. 20 is a bottom plan view of an electrode configuration for an implantable stimulation device in accordance with yet another embodiment of the invention.

FIG. 21 is a bottom plan view of an electrode configuration for an implantable stimulation device in accordance with another embodiment of the invention.

FIG. 22 is a bottom plan view of yet another embodiment of an electrode configuration for use with an implantable stimulation apparatus in accordance with the invention.

FIG. 23 is a bottom plan view and

FIG. 24a is a cross-sectional view of an electrode configuration for use with a stimulation apparatus in accordance with still another embodiment of the invention.

FIGS. 24b-c are bottom plan views of electrode configurations in accordance with still further embodiments of the invention.

FIG. 25 is an isometric view schematically illustrating a part of an implantable stimulation apparatus with a mechanical biasing element in accordance with an embodiment of the invention.

FIG. 26 is a cross-sectional view of a stimulation apparatus having a mechanical biasing element that has been implanted into a skull of a patient in accordance with an embodiment of the invention.

FIG. 27 is a cross-sectional view schematically illustrating a part of a stimulation apparatus having a biasing element in accordance with an embodiment of the invention.

FIG. 28 is a cross-sectional view of a stimulation apparatus having a biasing element in accordance with still another embodiment of the invention.

FIG. 29 is a cross-sectional view of a stimulation apparatus having a biasing element in accordance with yet another embodiment of the invention.

FIG. 30 is a cross-sectional view of a stimulation apparatus having a biasing element in accordance with yet another embodiment of the invention.

FIG. 31 is a cross-sectional view schematically illustrating a portion of an implantable stimulation apparatus having an external power source and pulse generator in accordance with an embodiment of the invention.

FIG. 32 is a cross-sectional view schematically illustrating a portion of an implantable stimulation apparatus having an external power source and pulse generator in accordance with another embodiment of the invention.

FIG. 33 is a cross-sectional view illustrating in greater detail a portion of the implantable stimulation apparatus of FIG. 32.

FIG. 34 is a cross-sectional view schematically illustrating a portion of an implantable stimulation apparatus and an external controller in accordance with another embodiment of the invention.

FIG. 35 is a cross-sectional view schematically illustrating a portion of an implantable stimulation apparatus and an external controller in accordance with yet another embodiment of the invention.

FIG. 36 is a cross-sectional view schematically illustrating a portion of an implantable stimulation apparatus in accordance with yet another embodiment of the invention.

FIG. 37 is an isometric view and

FIG. 38 is a cross-sectional view illustrating an implantable stimulation apparatus in accordance with an embodiment of the invention.

FIG. 39 is a cross-sectional view illustrating an implantable stimulation apparatus in accordance with yet another embodiment of the invention.

FIG. 40 is a schematic illustration of an implantable stimulation apparatus in accordance with an embodiment of the invention.

FIGS. 41A and 41B are schematic illustrations of an implanting procedure in accordance with an embodiment of the invention.

FIG. 42 is a cross-sectional view schematically illustrating electrical enhancement of cells implanted in the brain in accordance with another embodiment of the invention.

FIG. 43 is a cross-sectional view schematically illustrating electrical enhancement of cells implanted at or near the spinal cord or peripheral nerves in accordance with yet another embodiment of the invention.

FIG. 44 is a cross-sectional view schematically illustrating electrical enhancement of cells in vitro in accordance with still another embodiment of the invention.

### DETAILED DESCRIPTION

The following disclosure describes several methods and apparatus for electrical stimulation to treat or otherwise effectuate a change in neural-functions of a patient. For example, the following disclosure describes several methods for electrically stimulating cells implanted in the brain, spinal cord, and/or peripheral nerves of a patient. Methods in accordance with some embodiments of the invention are directed toward electrically enhancing the achievement of full functionality of cells capable of differentiating into neurons implanted in a patient's nervous system. Methods in accordance with other embodiments of the invention are directed toward electrically stimulating fully differentiated neurons implanted in a patient's nervous system to promote growth and connectivity of the implanted neurons.

Methods in accordance with further embodiments of the invention are directed toward enhancing or otherwise inducing neuroplasticity to effectuate a particular neural function. Neuroplasticity refers to the ability of the brain to change or adapt over time. It was once thought adult brains became relatively “hard wired” such that functionally significant neural networks could not change significantly over time or in response to injury. It has become increasingly more apparent that these neural networks can change and adapt over time so that meaningful function can be regained in response to brain injury. An aspect of several embodiments of methods in accordance with the invention is to provide the appropriate triggers for adaptive neuroplasticity. These appropriate triggers appear to cause or enable increased synchrony of functionally significant populations of neurons in a network.

Electrically enhanced or induced neural stimulation in accordance with several embodiments of the invention excites a portion of a neural network involved in a functionally significant task such that a selected population of neurons can become more strongly associated with that network. Because such a network will subserve a functionally meaningful task, such as motor relearning, the changes are more likely to be lasting because they are continually being reinforced by natural use mechanisms. The nature of stimulation in accordance with several embodiments of the invention ensures that the stimulated population of neurons links to other neurons in the functional network. It is expected that this occurs because action potentials are not actually caused by the stimulation, but rather are caused by interactions with other neurons in the network. Several aspects of the electrical stimulation in accordance with selected embodiments of the invention simply allows this to happen with an increased probability when the network is activated by favorable activities, such as rehabilitation or limb use.

The methods in accordance with the invention can be used to treat brain damage (e.g., stroke, trauma, etc.), brain disease (e.g., Alzheimer's, Pick's, Parkinson's, etc.), and/or brain disorders (e.g., epilepsy, depression, etc.). The methods in accordance with the invention can also be used to enhance functions of normal, healthy brains (e.g., learning, memory, etc.), or to control sensory functions (e.g., pain).

Certain embodiments of methods in accordance with the invention electrically stimulate the brain at a stimulation site where neuroplasticity is occurring. The stimulation site may be different than the region in the brain where neural activity is typically present to perform the particular function according to the functional organization of the brain. In one embodiment in which neuroplasticity related to the neural-function occurs in the brain, the method can include identifying the location where such neuroplasticity is present. This particular procedure may accordingly enhance a change in the neural activity to assist the brain in performing the particular neural function. In an alternative embodiment in which neuroplasticity is not occurring in the brain, an aspect is to induce neuroplasticity at a stimulation site where it is expected to occur. This particular procedure may thus induce a change in the neural activity to instigate performance of the neural function. Several embodiments of these methods are expected to produce a lasting effect on the intended neural activity at the stimulation site.

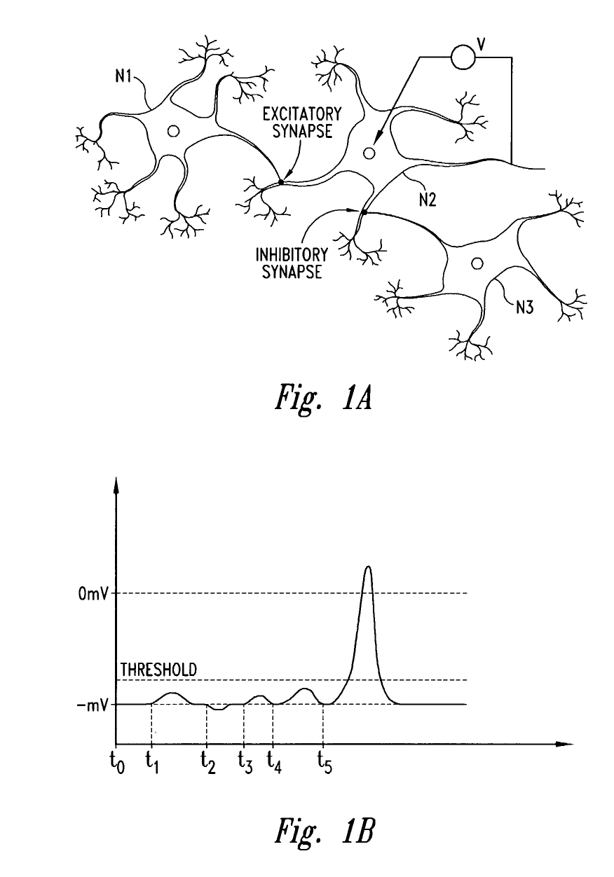

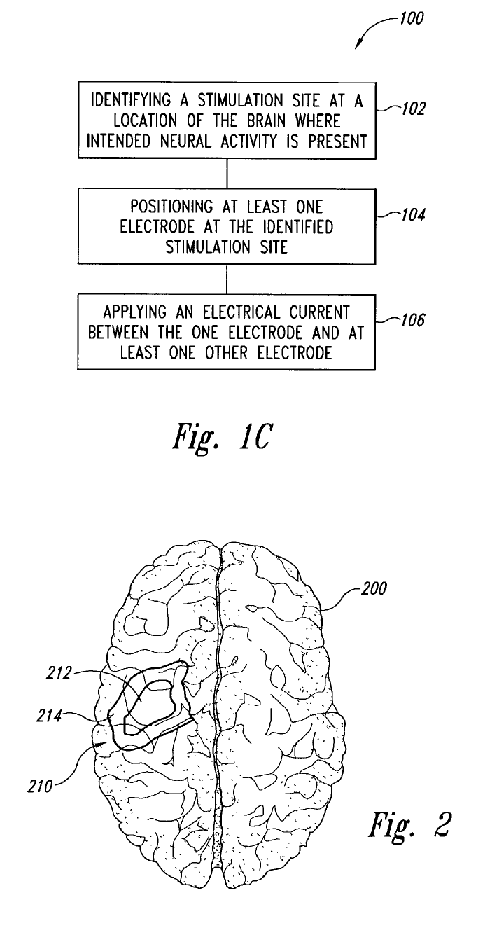

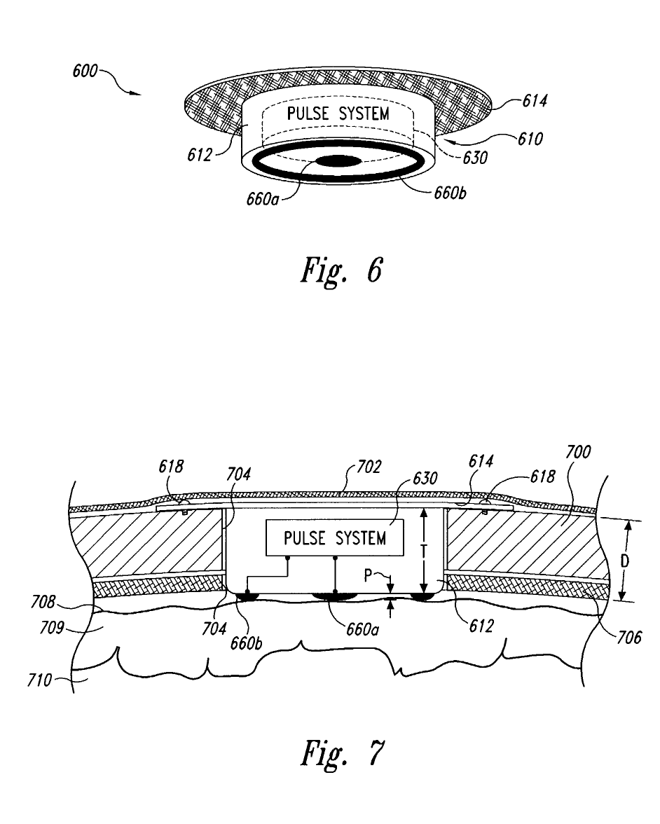

The specific details of certain embodiments of the invention are set forth in the following description and in FIGS. 1A-44 to provide a thorough understanding of these embodiments to a person of ordinary skill in the art. More specifically, several embodiments of methods in accordance with the invention are initially described with reference to FIGS. 1-5F. Several embodiments of devices for stimulating the central nervous system, including cortical and/or deep-brain regions of the brain, regions of the spinal cord, and peripheral nerves are described with reference to FIGS. 6-44. A person skilled in the art will understand that the present invention may have additional embodiments, and that the invention can be practiced without several of the details described below.

A. METHODS FOR ELECTRICALLY STIMULATING REGIONS OF THE BRAIN

1. Embodiments of Electrically Enhancing Neural Activity

FIG. 1A is a schematic representation of several neurons N1-N3 and FIG. 1B is a graph illustrating an “action potential” related to neural activity in a normal neuron. Neural activity is governed by electrical impulses generated in neurons. For example, neuron N1 can send excitatory inputs to neuron N2 (e.g., times t₁, t₃ and t₄ in FIG. 1B), and neuron N3 can send inhibitory inputs to neuron N2 (e.g., time t₂ in FIG. 1B). The neurons receive/send excitatory and inhibitory inputs from/to a population of other neurons. The excitatory and inhibitory inputs can produce “action potentials” in the neurons, which are electrical pulses that travel through neurons by changing the flux of sodium (Na) and potassium (K) ions across the cell membrane. An action potential occurs when the resting membrane potential of the neuron surpasses a threshold level. When this threshold level is reached, an “all-or-nothing” action potential is generated. For example, as shown in FIG. 1B, the excitatory input at time t₅causes neuron N2 to “fire” an action potential because the input exceeds the threshold level for generating the action potential. The action potentials propagate down the length of the axon (the long process of the neuron that makes up nerves or neuronal tracts) to cause the release of neurotransmitters from that neuron that will further influence adjacent neurons.

FIG. 1C is a flowchart illustrating a method 100 for effectuating a neural-function in a patient in accordance with an embodiment of the invention. The neural-function, for example, can control a specific mental process or physiological function, such as a particular motor function or sensory function (e.g., movement of a limb) that is normally associated with neural activity at a “normal” location in the brain according to the functional organization of the brain. In several embodiments of the method 100, at least some neural activity related to the neural-function can be occurring at a site in the brain. The site of the neural activity may be at the normal location where neural activity typically occurs to carry out the neural-function according to the functional organization of the brain, or the site of the neural activity may be at a different location where the brain has recruited material to perform the neural activity. In either situation, one aspect of several embodiments of the method 100 is to determine the location in the brain where this neural activity is present.

The method 100 includes a diagnostic procedure 102 involving identifying a stimulation site at a location of the brain where an intended neural activity related to the neural-function is present. In one embodiment, the diagnostic procedure 102 includes generating the intended neural activity in the brain from a “peripheral” location that is remote from the normal location, and then determining where the intended neural activity is actually present in the brain. In an alternative embodiment, the diagnostic procedure 102 can be performed by identifying a stimulation site where neural activity has changed in response to a change in the neural-function. The method 100 continues with an implanting procedure 104 involving positioning first and second electrodes at the identified stimulation site, and a stimulating procedure 106 involving applying an electrical current between the first and second electrodes. Many embodiments of the implanting procedure 104 position two or more electrodes at the stimulation site, but other embodiments of the implanting procedure involve positioning only one electrode at the stimulation site and another electrode remotely from the stimulation site. As such, the implanting procedure 104 of the method 100 can include implanting at least one electrode at the stimulation site. The procedures 102-106 are described in greater detail below.

FIGS. 2-4 illustrate an embodiment of the diagnostic procedure 102. The diagnostic procedure 102 can be used to determine the region of the brain where stimulation will likely effectuate the desired function, such as rehabilitating a loss of a neural-function caused by a stroke, trauma, disease or other circumstance. FIG. 2, more specifically, is an image of a normal, healthy brain 200 having a first region 210 where the intended neural activity occurs to effectuate a specific neural-function in accordance with the functional organization of the brain. For example, the neural activity in the first region 210 shown in FIG. 2 is generally associated with the movement of a patient's fingers. The first region 210 can have a high-intensity area 212 and a low-intensity area 214 in which different levels of neural activity occur. It is not necessary to obtain an image of the neural activity in the first region 210 shown in FIG. 2 to carry out the diagnostic procedure 102, but rather it is provided to show an example of neural activity that typically occurs at a “normal location” according to the functional organization of the brain 200 for a large percentage of people with normal brain function. It will be appreciated that the actual location of the first region 210 will generally vary between individual patients.

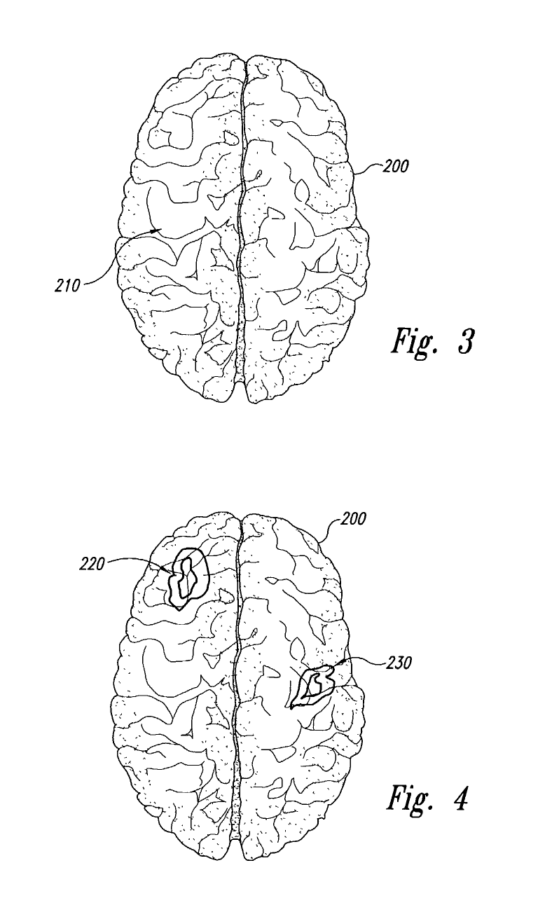

The neural activity in the first region 210, however, can be impaired. In a typical application, the diagnostic procedure 102 begins by taking an image of the brain 200 that is capable of detecting neural activity to determine whether the intended neural activity associated with the particular neural function of interest is occurring at the region of the brain 200 where it normally occurs according to the functional organization of the brain. FIG. 3 is an image of the brain 200 after the first region 210 has been affected (e.g., from a stroke, trauma or other cause). As shown in FIG. 3, the neural activity that controlled the neural-function for moving the fingers no longer occurs in the first region 210. The first region 210 is thus “inactive,” which is expected to result in a corresponding loss of the movement and/or sensation in the fingers. In some instances, the damage to the brain 200 may result in only a partial loss of the neural activity in the damaged region. In either case, the image shown in FIG. 3 establishes that the loss of the neural-function is related to the diminished neural activity in the first region 210. The brain 200 may accordingly recruit other neurons to perform neural activity for the affected neural-function (i.e., neuroplasticity), or the neural activity may not be present at any location in the brain.

FIG. 4 is an image of the brain 200 illustrating a plurality of potential stimulation sites 220 and 230 for effectuating the neural-function that was originally performed in the first region 210 shown in FIG. 2. FIGS. 3 and 4 show an example of neuroplasticity in which the brain compensates for a loss of neural-function in one region of the brain by recruiting other regions of the brain to perform neural activity for carrying out the affected neural-function. The diagnostic procedure 102 utilizes the neuroplasticity that occurs in the brain to identify the location of a stimulation site that is expected to be more responsive to the results of an electrical, magnetic, sonic, genetic, biologic, and/or pharmaceutical procedure to effectuate the desired neural-function.

One embodiment of the diagnostic procedure 102 involves generating the intended neural activity remotely from the first region 210 of the brain, and then detecting or sensing the location in the brain where the intended neural activity has been generated. The intended neural activity can be generated by applying an input that causes a signal to be sent to the brain. For example, in the case of a patient that has lost the use of limb, the affected limb is moved and/or stimulated while the brain is scanned using a known imaging technique that can detect neural activity (e.g., functional MRI, positron emission tomography, etc.). In one specific embodiment, the affected limb can be moved by a practitioner or the patient, stimulated by sensory tests (e.g., pricking), or subject to peripheral electrical stimulation. The movement/stimulation of the affected limb produces a peripheral neural signal from the limb that is expected to generate a response neural activity in the brain. The location in the brain where this response neural activity is present can be identified using the imaging technique. FIG. 4, for example, can be created by moving the affected fingers and then noting where neural activity occurs in response to the peripheral stimulus. By peripherally generating the intended neural activity, this embodiment may accurately identify where the brain has recruited matter (i.e., sites 220 and 230) to perform the intended neural activity associated with the neural-function.

An alternative embodiment of the diagnostic procedure 102 involves identifying a stimulation site at a second location of the brain where the neural activity has changed in response to a change in the neural-function of the patient. This embodiment of the method does not necessarily require that the intended neural activity be generated by peripherally actuating or stimulating a body part. For example, the brain can be scanned for neural activity associated with the impaired neural-function as a patient regains use of an affected limb or learns a task over a period of time. This embodiment, however, can also include peripherally generating the intended neural activity remotely from the brain explained above.

In still another embodiment, the diagnostic procedure 102 involves identifying a stimulation site at a location of the brain where the intended neural activity is developing to perform the neural-function. This embodiment is similar to the other embodiments of the diagnostic procedure 102, but it can be used to identify a stimulation site at (a) the normal region of the brain where the intended neural activity is expected to occur according to the functional organization of the brain and/or (b) a different region where the neural activity occurs because the brain is recruiting additional matter to perform the neural-function. This particular embodiment of the method involves monitoring neural activity at one or more locations where the neural activity occurs in response to the particular neural-function of interest. For example, to enhance the ability to learn a particular task (e.g., playing a musical instrument, memorizing, etc.), the neural activity can be monitored while a person performs the task or thinks about performing the task. The stimulation sites can be defined by the areas of the brain where the neural activity has the highest intensity, the greatest increases, and/or other parameters that indicate areas of the brain that are being used to perform the particular task.

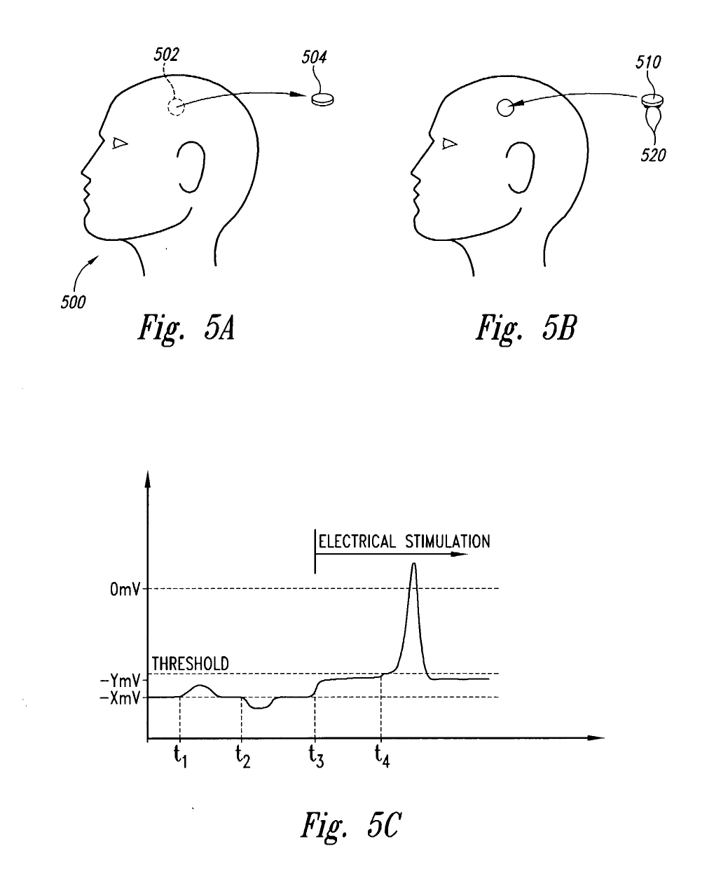

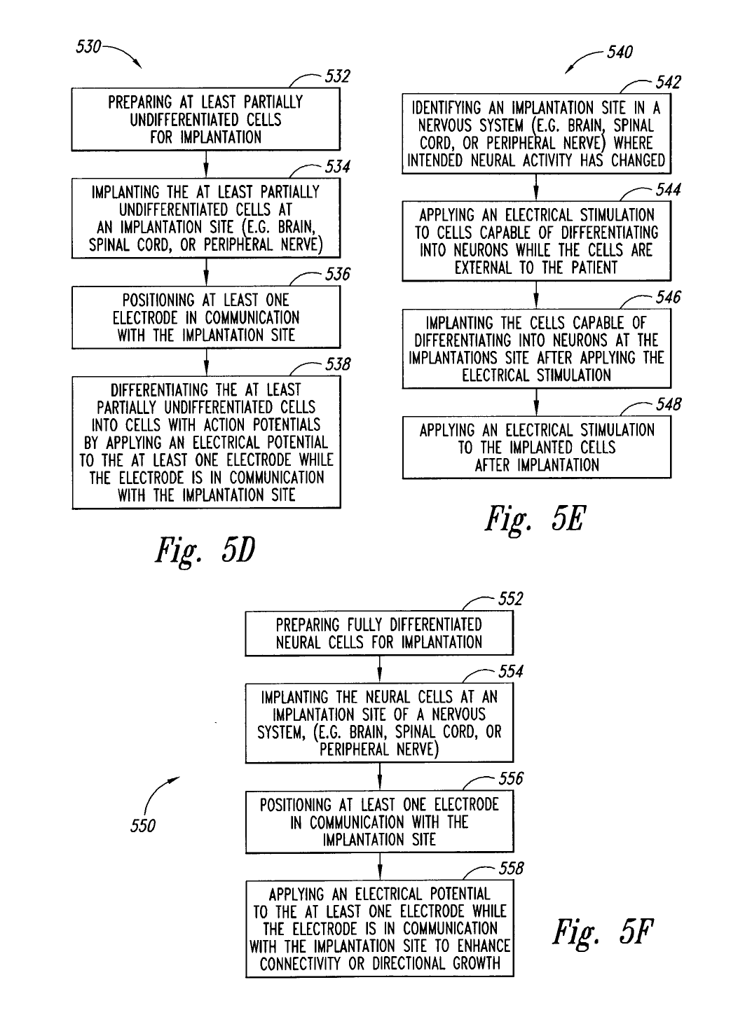

FIGS. 5A and 5B are schematic illustrations of the implanting procedure 104 described above with reference to FIG. 1C for positioning the first and second electrodes relative to a portion of the brain of a patient 500. Referring to FIG. 5A, a stimulation site 502 is identified in accordance with an embodiment of the diagnostic procedure 102. In one embodiment, a skull section 504 is removed from the patient 500 adjacent to the stimulation site 502. The skull section 504 can be removed by boring a hole in the skull in a manner known in the art, or a much smaller hole can be formed in the skull using drilling techniques that are also known in the art. In general, the hole can be 0.2-4.0 cm in diameter. Referring to FIG. 5B, an implantable stimulation apparatus 510 having first and second electrodes 520 can be implanted in the patient 500. Suitable techniques associated with the implantation procedure are known to practitioners skilled in the art. After the stimulation apparatus 510 has been implanted in the patient 500, a pulse system generates electrical pulses that are transmitted to the stimulation site 502 by the first and second electrodes 520. Stimulation apparatus suitable for carrying out the foregoing embodiments of methods in accordance with the invention are described in more detail below with reference to the FIGS. 6-40.

Several embodiments of methods for enhancing neural activity in accordance with the invention are expected to provide lasting results that promote the desired neural-function. Before the present invention, electrical and magnetic stimulation techniques typically stimulated the normal locations of the brain where neural activity related to the neural-functions occurred according to the functional organization of the brain. Such conventional techniques, however, may not be effective because the neurons in the “normal locations” of the brain may not be capable of carrying out the neural activity because of brain damage, disease, disorder, and/or because of variations of the location specific to individual patients. Several embodiments of methods for enhancing neural activity in accordance with the invention overcome this drawback by identifying a stimulation site based on neuroplastic activity that appears to be related to the neural-function. By first identifying a location in the brain that is being recruited to perform the neural activity, it is expected that therapies (e.g., electrical, magnetic, genetic, biologic, and/or pharmaceutical) applied to this location will be more effective than conventional techniques. This is because the location that the brain is recruiting for the neural activity may not be the “normal location” where the neural activity would normally occur according to the functional organization of the brain. Therefore, several embodiments of methods for enhancing neural activity in accordance with the invention are expected to provide lasting results because the therapies are applied to the portion of the brain where neural activity for carrying out the neural-function actually occurs in the particular patient.

2. Electrically Inducing Desired Neural Activity

The method 100 for effectuating a neural-function can also be used to induce neural activity in a region of the brain where such neural activity is not present. As opposed to the embodiments of the method 100 described above for enhancing existing neural activity, the embodiments of the method 100 for inducing neural activity initiate the neural activity at a stimulation site where it is estimated that neuroplasticity will occur. In this particular situation, an image of the brain seeking to locate where neuroplasticity is occurring may be similar to FIG. 3. An aspect of inducing neural activity, therefore, is to develop a procedure to determine where neuroplasticity is likely to occur.

A stimulation site may be identified by estimating where the brain will likely recruit neurons for performing the neural-function. In one embodiment, the location of the stimulation site is estimated by defining a region of the brain that is proximate to the normal location where neural activity related to the neural-function is generally present according to the functional organization of the brain. An alternative embodiment for locating the stimulation site includes determining where neuroplasticity has typically occurred in patients with similar symptoms. For example, if the brain typically recruits a second region of the cortex to compensate for a loss of neural activity in the normal region of the cortex, then the second region of the cortex can be selected as the stimulation site either with or without imaging the neural activity in the brain.

Several embodiments of methods for inducing neural activity in accordance with the invention are also expected to provide lasting results that initiate and promote a desired neural-function. By first estimating the location of a stimulation site where desired neuroplasticity is expected to occur, therapies applied to this location may be more effective than conventional therapies for reasons that are similar to those explained above regarding enhancing neural activity. Additionally, methods for inducing neural activity may be easier and less expensive to implement because they do not require generating neural activity and/or imaging the brain to determine where the intended neural activity is occurring before applying the therapy.

3. Applications of Methods for Electrically Stimulating Regions of the Brain

The foregoing methods for enhancing existing neural activity or inducing new neural activity are expected to be useful for many applications. As explained above, several embodiments of the method 100 involve determining an efficacious location of the brain to enhance or induce an intended neural activity that causes the desired neural-functions to occur. Additional therapies can also be implemented in combination with the electrical stimulation methods described above. Several specific applications using embodiments of electrical stimulation methods in accordance with the invention either alone or with adjunctive therapies will now be described, but it will be appreciated that the methods in accordance with the invention can be used in many additional applications.

a. General Applications

The embodiments of the electrical stimulation methods described above are expected to be particularly useful for rehabilitating a loss of mental functions, motor functions and/or sensory functions caused by damage to the brain. In a typical application, the brain has been damaged by a stroke or trauma (e.g., automobile accident). The extent of the particular brain damage can be assessed using functional MRI or another appropriate imaging technique as explained above with respect to FIG. 3. A stimulation site can then be identified by: (a) peripherally stimulating a body part that was affected by the brain damage to induce the intended neural activity and determining the location where a response neural activity occurs; (b) determining where the neural activity has changed as a patient gains more use of the affected body part; and/or (c) estimating the location that the brain may recruit neurons to carry out the neural activity that was previously performed by the damaged portion of the brain. An electrical stimulation therapy can then be applied to the selected stimulation site by placing the first and second electrodes relative to the stimulation site to apply an electrical current in that portion of the brain. As explained in more detail below, it is expected that applying an electrical current to the portion of the brain that has been recruited to perform the neural activity related to the affected body part will produce a lasting neurological effect for rehabilitating the affected body part.

Several specific applications are expected to have a stimulation site in the cortex because neural activity in this part of the brain effectuates motor functions and/or sensory functions that are typically affected by a stroke or trauma. In these applications, the electrical stimulation can be applied directly to the pial surface of the brain or at least proximate to the pial surface (e.g., the dura mater, the fluid surrounding the cortex, or neurons within the cortex). Suitable devices for applying the electrical stimulation to the cortex are described in detail with reference to FIGS. 6-42.

The electrical stimulation methods can also be used with adjunctive therapies to rehabilitate damaged portions of the brain. In one embodiment, the electrical stimulation methods can be combined with physical therapy and/or drug therapies to rehabilitate an affected neural function. For example, if a stroke patient has lost the use of a limb, the patient can be treated by applying the electrical therapy to a stimulation site where the intended neural activity is present while the affected limb is also subject to physical therapy. An alternative embodiment can involve applying the electrical therapy to the stimulation site and chemically treating the patient using amphetamines or other suitable drugs.

The embodiments of the electrical stimulation methods described above are also expected to be useful for treating brain diseases, such as Alzheimer's, Parkinson's, and other brain diseases. In this application, the stimulation site can be identified by monitoring the neural activity using functional MRI or other suitable imaging techniques over a period of time to determine where the brain is recruiting material to perform the neural activity that is being affected by the disease. It may also be possible to identify the stimulation site by having the patient try to perform an act that the particular disease has affected, and monitoring the brain to determine whether any response neural activity is present in the brain. After identifying where the brain is recruiting additional matter, the electrical stimulation can be applied to this portion of the brain. It is expected that electrically stimulating the regions of the brain that have been recruited to perform the neural activity which was affected by the disease will assist the brain in offsetting the damage caused by the disease.

The embodiments of the electrical stimulation methods described above are also expected to be useful for treating neurological disorders, such as depression, passive-aggressive behavior, weight control, and other disorders. In these applications, the electrical stimulation can be applied to a stimulation site in the cortex or another suitable part of the brain where neural activity related to the particular disorder is present. The embodiments of electrical stimulation methods for carrying out the particular therapy can be adapted to either increase or decrease the particular neural activity in a manner that produces the desired results. For example, an amputee may feel phantom sensations associated with the amputated limb. This phenomenon can be treated by applying an electrical pulse that reduces the phantom sensations. The electrical therapy can be applied so that it will modulate the ability of the neurons in that portion of the brain to execute sensory functions.

b. Pulse Forms and Potentials

The electrical stimulation methods in accordance with the invention can use several different pulse forms to effectuate the desired neuroplasticity. The pulses can be a bi-phasic or monophasic stimulus that is applied to achieve a desired potential in a sufficient percentage of a population of neurons at the stimulation site. In one embodiment, the pulse form has a frequency of approximately 1-1000 Hz, but the frequency may be particularly useful in the range of approximately 20-200 Hz. For example, initial clinical trials are expected to use a frequency of approximately 50-100 Hz. The pulses can also have pulse widths of approximately 10 μs-100 ms, or more specifically the pulse width can be approximately 20-200 μs. For example, a pulse width of 50-100 μs may produce beneficial results.

It is expected that one particularly useful application of the invention involves enhancing or inducing neuroplasticity by raising the resting membrane potential of neurons to bring the neurons closer to the threshold level for firing an action potential. Because the stimulation raises the resting membrane potential of the neurons, it is expected that these neurons are more likely to “fire” an action potential in response to excitatory input at a lower level.

FIG. 5C is a graph illustrating applying a subthreshold potential to the neurons N1-N3 of FIG. 1A. At times t₁ and t₂, the excitatory/inhibitory inputs from other neurons do not “bridge-the-gap” from the resting potential at −X mV to the threshold potential. At time t₃, the electrical stimulation is applied to the brain to raise the resting potential of neurons in the stimulated population such that the resting potential is at −Y mV. As such, at time t₄ when the neurons receive another excitatory input, even a small input exceeds the gap between the raised resting potential −Y mV and the threshold potential to induce action potentials in these neurons. For example, if the resting potential is approximately −70 mV and the threshold potential is approximately −50 mV, then the electrical stimulation can be applied to raise the resting potential of a sufficient number of neurons to approximately −52 to −60 mV.

The actual electrical potential applied to electrodes implanted in the skull to achieve a subthreshold potential stimulation will vary according to the individual patient, the type of therapy, the type of electrodes, and other factors. In general, the pulse form of the electrical stimulation (e.g., the frequency, pulse width, wave form, and voltage potential) is selected to raise the resting potential in a sufficient number of neurons at the stimulation site to a level that is less than a threshold potential for a statistical portion of the neurons in the population. The pulse form, for example, can be selected so that the applied voltage of the stimulus achieves a change in the resting potential of approximately 10%-95%, of the difference between the unstimulated resting potential and the threshold potential. In specific embodiments, the stimulus can achieve a change of 60-80% or 50-80% of the difference between the unstimulated resting potential and the threshold potential.

In other embodiments, the voltage level of the stimulus can be selected independent of neuron resting potential. For example, the stimulus can be selected to be some value less than the threshold for generating an action potential. In one embodiment, the voltage value can be from about 10% to about 60% less than the threshold for generating an action potential. In other embodiments, this range can have other values, such as from about 10% to about 50%, from about 20% to about 50%, and from about 30% to about 60% less than the threshold for generating an action potential.

In one specific example of a subthreshold application for treating a patient's hand, electrical stimulation is not initially applied to the stimulation site. Although physical therapy related to the patient's hand may cause some activation of a particular population of neurons that is known to be involved in “hand function,” only a low level of activation might occur because physical therapy only produces a low level of action potential generation in that population of neurons. However, when the subthreshold electrical stimulation is applied, the resting membrane potentials of the neurons in the stimulated population are elevated. These neurons now are much closer to the threshold for action potential formation such that when the same type of physical therapy is given, this population of cells will have a higher level of activation because these cells are more likely to fire action potentials.

Subthreshold stimulation may produce better results than simply stimulating the neurons with sufficient energy levels to exceed the threshold for action potential formation. One aspect of subthreshold stimulation is to increase the probability that action potentials will occur in response to the ordinary causes of activation—such as physical therapy. This will allow the neurons in this functional network to become entrained together, or “learn” to become associated with these types of activities. If neurons are given so much electricity that they continually fire action potentials without additional excitatory inputs (suprathreshold stimulation), this will create “noise” and disorganization that will not likely cause improvement in function. In fact, neurons that are “overdriven” soon deplete their neurotransmitters and effectively become silent.

The application of a subthreshold stimulation is very different than suprathreshold stimulation. Subthreshold stimulation in accordance with several embodiments of the invention, for example, does not intend to directly make neurons fire action potentials with the electrical stimulation in a significant population of neurons at the stimulation site. Instead, subthreshold stimulation attempts to decrease the “activation energy” required to activate a large portion of the neurons at the stimulation site. As such, subthreshold stimulation in accordance with certain embodiments of the invention is expected to increase the probability that the neurons will fire in response to the usual intrinsic triggers, such as trying to move a limb, physical therapy, or simply thinking about movement of a limb, etc. Moreover, coincident stimulation associated with physical therapy is expected to increase the probability that the action potentials that are occurring with an increased probability due to the subthreshold stimulation will be related to meaningful triggers, and not just “noise.”

The stimulus parameters set forth above, such as a frequency selection of approximately 50-100 Hz and an amplitude sufficient to achieve an increase of 50% to 80% of the difference between the resting potential and the threshold potential are specifically selected so that they will increase the resting membrane potential of the neurons, thereby increasing the likelihood that they will fire action potentials, without directly causing action potentials in most of the neuron population. In addition, and as explained in more detail later with respect to FIGS. 6-42, several embodiments of stimulation apparatus in accordance with the invention are designed to precisely apply a pulse form that produces subthreshold stimulation by selectively stimulating regions of the cerebral cortex of approximately 1-2 cm (the estimated size of a “functional unit” of cortex), directly contacting the pial surface or the dural surface with the electrodes to consistently create the same alterations in resting membrane potential, and/or biasing the electrodes against the pial surface (or dural surface) to provide a positive connection between the electrodes and the cortex.

As is discussed immediately below with reference to FIGS. 5D-F, many or all of the foregoing techniques can be applied to implanted cells to affect the functionality, growth, and/or development of such cells.

B. METHODS FOR ELECTRICAL STIMULATION OF CELLS IMPLANTED IN THE NERVOUS SYSTEM

FIG. 5D is a flowchart illustrating a method 530 for electrically stimulating cells implanted in the nervous system of a patient in accordance with an embodiment of the invention. The method 530 can include a preparation procedure 532 including preparing at least partially undifferentiated cells for implantation. Suitable techniques for preparing the cells for implantation are generally known to practitioners skilled in the art. An at least partially undifferentiated cell is defined as a cell capable of differentiating or further differentiating from an initial state into a cell (such as a neuron) that exhibits increased action potential characteristics when compared to the cell in its initial state. At least partially undifferentiated cells can include stem cells, progenitor cells, and precursor cells. Stem cells are characterized as being completely undifferentiated; they can divide without limit, and when they divide each daughter cell can remain a stem cell, or assume the physical and/or functional characteristics of a cell that it is replacing. For example, stem cells are capable of differentiating into neurons or glial cells. In one embodiment, a progenitor cell can be partially undifferentiated and can therefore have a more restricted potential cellular purpose than a stem cell. For example, some progenitor cells may only develop into neurons or glia. In one embodiment, a precursor cell can be even more differentiated than a progenitor cell and can have even more restricted cellular purposes. For example, a neuroblast can only become a neuron. In other embodiments, the at least partially undifferentiated cells can include other cell types. Accordingly, stem cells, progenitor cells and precursor cells are representative examples rather than an exhaustive list of at least partially undifferentiated cells.

The method 530 can further include an implantation procedure 534 involving implanting the at least partially undifferentiated cells at an identified implantation site, e.g. a portion of the brain, spinal cord, or peripheral nerve. In one embodiment, the implantation site in the brain can be identified by performing the diagnostic procedure 102 described above with reference to FIGS. 2-4. In other embodiments, the implantation site can be identified by other techniques, for example, electrodiagnostic procedures such as stimulating the spinal cord and observing electrophysiological and functional responses. Alternatively, anatomical identification techniques can be used to identify regions of the spinal cord or a peripheral nerve that may have suffered from damage, disease or disorder. Common methods for implanting the cells include injecting the cells into the implantation site with a delivery device such as a tube, catheter, and/or syringe. Apparatus and methods suitable for implanting the cells are described in more detail below with reference to FIGS. 41A and 41B.

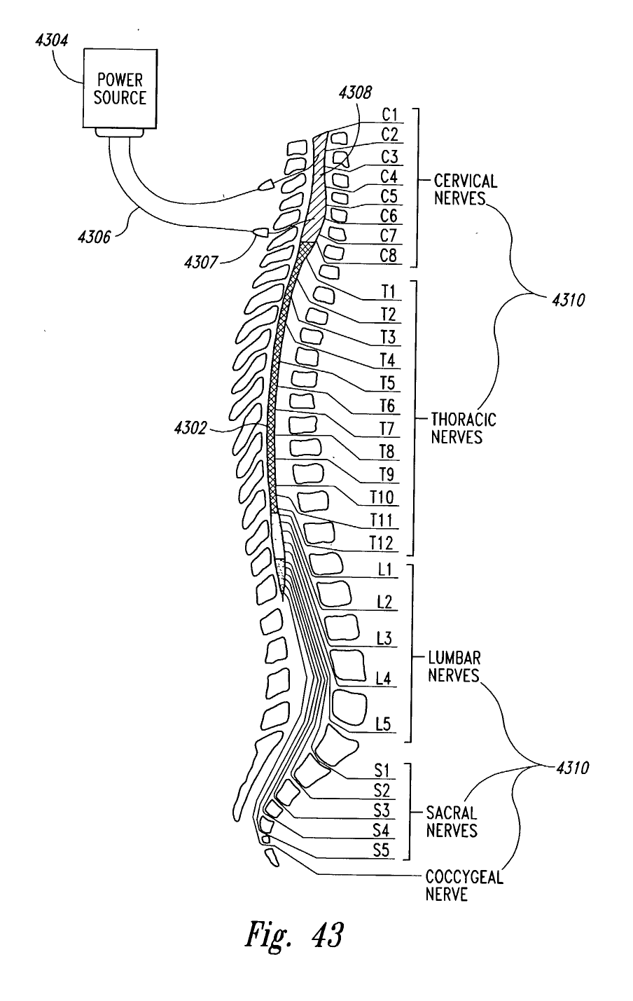

The method 530 can still further include a positioning procedure 536 involving positioning at least one electrode in communication with the implantation site. In one embodiment, at least one electrode is positioned at least proximate to the implantation site. For example, when the cells are implanted in the brain, the electrode(s) can be positioned in a manner generally similar to that described above with reference to FIGS. 5A and 5B. When the cells are implanted in or at least proximate to the spinal cord or a peripheral nerve, the electrode(s) can be positioned in accordance with methods described below with reference to FIG. 43.

In other embodiments, the relative position between the at least one electrode and the implantation site can be different, while still allowing for communication between the electrode and the implantation site. For example, the electrode may be positioned to directly stimulate cells in the cortex, and affect (via the stimulation of cortical cells) implanted cells deep in the brain. In one aspect of this embodiment, the implanted cells can be stimulated through physical/electrical connections with cortical neurons. In another aspect of this embodiment, the implanted cells can be stimulated via factors such as growth factors produced by the cells immediately proximate to the electrode. Accordingly, the electrode can directly stimulate a “native” cell proximate to it and, via the native cell, provide stimulation to a more distant implanted cell.

The method 530 can further include a stimulation procedure 540 involving differentiating the at least partially undifferentiated cells into cells with increased action potentials by applying an electrical potential to the at least one electrode while the electrode is in communication with the implantation site. As described above, the electrode can be proximate to the implantation site, or can communicate with the implantation site (and cells at the implantation site) via other cells, such as native cells. Stimulation apparatus suitable for carrying out the foregoing embodiment of method 530 in accordance with the invention are described in more detail below with reference to FIGS. 6-44.

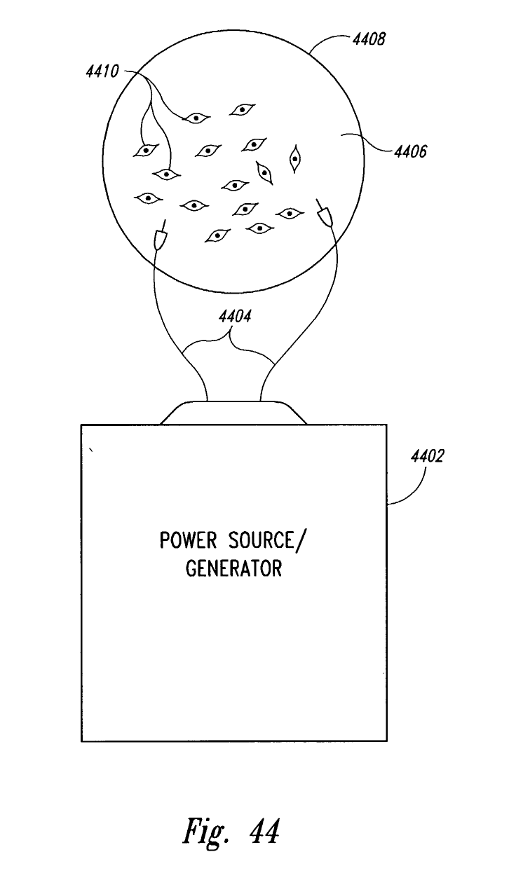

FIG. 5E is a flow chart illustrating another method 540 for electrically stimulating cells implanted in the nervous system of a patient in accordance with another embodiment of the invention. The method 540 can include an identification procedure 542 involving identifying an implantation site in a nervous system of a patient where the intended neural activity has changed. For example, the identification procedure 542 can include identifying an implantation site in a manner generally similar to any of those described above with reference to FIGS. 2-4. The method 540 can further include a stimulation procedure 544 involving applying an electrical stimulation to cells capable of differentiating into neurons while the cells are external to the patient. Apparatus suitable for carrying out the foregoing procedure are described in more detail below with reference to FIG. 44. After applying an electrical stimulation to the cells, the method 540 can continue with an implantation procedure 546 that includes implanting the cells into tissue at the implantation site of the nervous system of the patient. Optionally, the method can include applying further electrical stimulation to the cells after the cells have been implanted (step 548).

FIG. 5F is a flow chart illustrating another method 550 for electrically stimulating cells implanted in the nervous system of a patient in accordance with another embodiment of the invention. The method 550 can include a preparation procedure 552 involving preparing fully differentiated neural cells for implantation. The preparation procedure 552 can include applying electrical stimulation to the fully differentiated cells while the cells are external to the patient. The method 550 can continue with an implantation procedure 554 that includes implanting the fully differentiated neural cells at an implantation site of the nervous system, such as the brain, spinal cord, or a peripheral nerve. After implanting the neural cells, the method 550 can continue with a positioning procedure 556 involving positioning at least one electrode in communication with the implantation site. The method 550 can further include a stimulation procedure 558 involving applying electrical potential to the at least one electrode while the electrode is in communication with the implantation site. Accordingly, the electrical stimulation can provide spatial orientation information to the fully differentiated neural cells causing them to grow along preferred pathways or in preferred spatial orientations. The electrical stimulation can also help to generate further connectivity between neural cells, i.e., connections between neurons within the neural network. Such connections are required for the neural cells to transmit neural signals.

Implanted cells assume many physical and functional characteristics of the surrounding native cells. However, the process of implanting cells alone often does not achieve full functionality of the implanted cells. Electrical stimulation before and/or after implantation may enhance the ability of the implanted cells to achieve full functionality and growth. Accordingly, electrical stimulation may be particularly suitable for cells (such as neurons) that generate action potentials when functional. In one embodiment, the cells are electrically stimulated throughout the entire course of the differentiation process. In another embodiment, the cells are stimulated only after they reach a specific stage of development, for example, when the cells develop to the point of exhibiting action potentials. In still a further embodiment, the amplitude of the stimulation signal may be adjusted to excite action potentials in the adjacent native cells, even though the implanted cells may not initially exhibit action potentials themselves. Alternatively, the adjacent native cells may be stimulated at sub-threshold amplitudes. In other embodiments, stimulation may be continuous or intermittent during the cell differentiation process. In any of these embodiments, the presence of either sub-threshold or supra-threshold electrical stimulation is believed to enhance, guide, and/or promote the growth and/or functionality of the implanted cells.

Implanted cells, whether undifferentiated, fully differentiated or partially differentiated, may fail to grow in the directions required to complete functional connections with other cells. Accordingly, electrical stimulation may provide the necessary directional information to developing cells to increase their connectivity to neighboring cells. Growth of the implanted cells can be directed by specifically orienting the electrical stimulation such that the cell grows toward a targeted region and/or toward another neural cell. In one embodiment, relatively broad stimulation of the tissue surrounding the implanted cells orients the cells and increases the likelihood. of making connections with neighboring cells. In an alternative embodiment, electrodes can be positioned to direct electrical stimulation to an intermediate region between the implanted cells and the targeted region. In any of these embodiments, electrical stimulation is believed to align polarized molecules to direct growth and/or enhance effects that impact growth, such as the generation of growth factors, increased circulation, and/or increased production of neurotransmitters. As a result, the functionality of the implanted cells can be improved.

In other embodiments, the process of electrically stimulating cells before and/or after implantation can include other steps. For example, it is well known that migrating and developing cells respond to a number of factors, such as cell surface proteins and growth factors. Accordingly, the electrical stimulation techniques described above may be accompanied by a regimen of appropriate drugs to encourage the development of cell surface proteins and/or growth factors in the implanted cells.

C. DEVICES FOR ELECTRICALLY STIMULATING REGIONS OF THE BRAIN

FIGS. 6-40 illustrate stimulation apparatus in accordance with several embodiments of the invention for electrically stimulating regions of the brain in accordance with one or more of the methods described above. The devices illustrated in FIGS. 6-40 are generally used to stimulate a region of the cortex proximate to the pial surface of the brain (e.g., the dura mater, the pia mater, the fluid between the dura mater and the pia mater, and a depth in the cortex outside of the white matter of the brain). The devices can also be adapted for stimulating other portions of the brain in other embodiments.

1. Implantable Stimulation Apparatus With Integrated Pulse Systems

FIG. 6 is an isometric view and FIG. 7 is a cross-sectional view of a stimulation apparatus 600 in accordance with an embodiment of the invention for stimulating a region of the cortex proximate to the pial surface. In one embodiment, the stimulation apparatus 600 includes a support member 610, an integrated pulse-system 630 (shown schematically) carried by the support member 610, and first and second electrodes 660 (identified individually by reference numbers 660a and 660b). The first and second electrodes 660 are electrically coupled to the pulse system 630. The support member 610 can be configured to be implanted into the skull or another intracranial region of a patient. In one embodiment, for example, the support member 610 includes a housing 612 and an attachment element 614 connected to the housing 612. The housing 612 can be a molded casing formed from a biocompatible material that has an interior cavity for carrying the pulse system 630. The housing can alternatively be a biocompatible metal or another suitable material. The housing 612 can have a diameter of approximately 1-4 cm, and in many applications the housing 612 can be 1.5-2.5 cm in diameter. The housing 612 can also have other shapes (e.g., rectilinear, oval, elliptical) and other surface dimensions. The stimulation apparatus 600 can weigh 35g or less and/or occupy a volume of 20 cc or less. The attachment element 614 can be a flexible cover, a rigid plate, a contoured cap, or another suitable element for holding the support member 610 relative to the skull or other body part of the patient. In one embodiment, the attachment element 614 is a mesh, such as a biocompatible polymeric mesh, metal mesh, or other suitable woven material. The attachment element 614 can alternatively be a flexible sheet of Mylar, a polyester, or another suitable material.

FIG. 7, more specifically, is a cross-sectional view of the stimulation apparatus 600 after it has been implanted into a patient in accordance with an embodiment of the invention. In this particular embodiment, the stimulation apparatus 600 is implanted into the patient by forming an opening in the scalp 702 and cutting a hole 704 through the skull 700 and through the dura mater 706. In another embodiment, the hole 704 does not extend through the dura mater 706. In either embodiment, the hole 704 can be sized to receive the housing 612 of the support member 610, and in most applications, the hole 704 should be smaller than the attachment element 614. A practitioner inserts the support member 610 into the hole 704 and then, secures the attachment element 614 to the skull 700. The attachment element 614 can be secured to the skull using a plurality of fasteners 618 (e.g., screws, spikes, etc.) or an adhesive. In an alternative embodiment, a plurality of downwardly depending spikes can be formed integrally with the attachment element 614 to define anchors that can be driven into the skull 700.

The embodiment of the stimulation apparatus 600 shown in FIG. 7 is configured to be implanted into a patient so that the electrodes 660 contact a desired portion of the dura member 706 or the pia mater 708 at the stimulation site. For example, the housing 612 and the electrodes 660 can project from the attachment element 614 by a distance “D” such that the electrodes 660 are positioned at least proximate to the pia mater 708 surrounding the cortex 709. In another embodiment, the housing 612 and the electrodes 660 can project by a distance less than “D” to be positioned at least proximate to the pia mater 706. In either embodiment, the electrodes 660 can project from a housing 612 as shown in FIG. 7, or the electrodes 660 can be flush with the interior surface of the housing 612. In the particular embodiment shown in FIG. 7, the housing 612 has a thickness “T” and the electrodes 660 project from the housing 612 by a distance “P” so that the electrodes 660 press against the surface of the pia mater 708. The thickness of the housing 612 can be approximately 0.5-4 cm, and is more generally about 1-2 cm. The configuration of the stimulation apparatus 600 is not limited to the embodiment shown in FIGS. 6 and 7, but rather the housing 612, the attachment element 614, and the electrodes 660 can be configured to position the electrodes in several different regions of the brain. For example, in an alternate embodiment, the housing 612 and the electrodes 660 can be configured to position the electrodes deep within the cortex 709, and/or a deep brain region 710. In general, the electrodes can be flush with the housing or extend 0.1 mm to 5 cm from the housing. More specific embodiments of pulse system and electrode configurations for the stimulation apparatus will be described below.

Several embodiments of the stimulation apparatus 600 are expected to be more effective than existing transcranial electrical stimulation devices and transcranial magnetic stimulation devices. It will be appreciated that much of the power required for transcranial therapies is dissipated in the scalp and skull before it reaches the brain. In contrast to conventional transcranial stimulation devices, the stimulation apparatus 600 is implanted so that the electrodes are at least proximate to the pial surface or the dural surface of the brain 708. Several embodiments of methods in accordance with the invention can use the stimulation apparatus 600 to apply an electrical therapy directly to the pia mater 708, the dura mater 706, and/or another portion of the cortex 709 at significantly lower power levels than existing transcranial therapies. For example, a potential of approximately 1 mV to 10 V can be applied to the electrodes 660; in many instances a potential of 100 mV to 5 V can be applied to the electrodes 660 for selected applications. It will also be appreciated that other potentials can be applied to the electrodes 660 of the stimulation apparatus 600 in accordance with other embodiments of the invention.

Selected embodiments of the stimulation apparatus 600 are also capable of applying stimulation to a precise stimulation site. Again, because the stimulation apparatus 600 positions the electrodes 660 at least proximate to the pia mater 708 or the dura mater 706, precise levels of stimulation with good pulse shape fidelity will be accurately transmitted to the stimulation site in the brain. It will be appreciated that transcranial therapies may not be able to apply stimulation to a precise stimulation site because the magnetic and electrical properties of the scalp and skull may vary from one patient to another such that an identical stimulation by the transcranial device may produce a different level of stimulation at the neurons in each patient. Moreover, the ability to focus the stimulation to a precise area is hindered by delivering the stimulation transcranially because the scalp, skull and dura all diffuse the energy from a transcranial device. Several embodiments of the stimulation apparatus 600 overcome this drawback because the electrodes 660 are positioned under the skull 700 such that the pulses generated by the stimulation apparatus 600 are not diffused by the scalp 702 and skull 700.

2. Integrated Pulse Systems for Implantable Stimulation Apparatus

The pulse system 630 shown in FIGS. 6 and 7 generates and/or transmits electrical pulses to the electrodes 660 to create an electrical field at a stimulation site in a region of the brain. The particular embodiment of the pulse system 630 shown in FIG. 7 is an “integrated” unit in that is carried by the support member 610. The pulse system 630, for example, can be housed within the housing 612 so that the electrodes 660 can be connected directly to the pulse system 630 without having leads outside of the stimulation apparatus 600. The distance between the electrodes 660 and the pulse system 630 can be less than 4 cm, and it is generally 0.10 to 2.0 cm. The stimulation apparatus 600 can accordingly provide electrical pulses to the stimulation site without having to surgically create tunnels running through the patient to connect the electrodes 660 to a pulse generator implanted remotely from the stimulation apparatus 600. It will be appreciated, however, that alternative embodiments of stimulation apparatus in accordance with the invention can include a pulse system implanted separately from the stimulation apparatus 600 in the cranium or an external pulse system. Several particular embodiments of pulse systems that are suitable for use with the stimulation apparatus 600 will now be described in more detail.

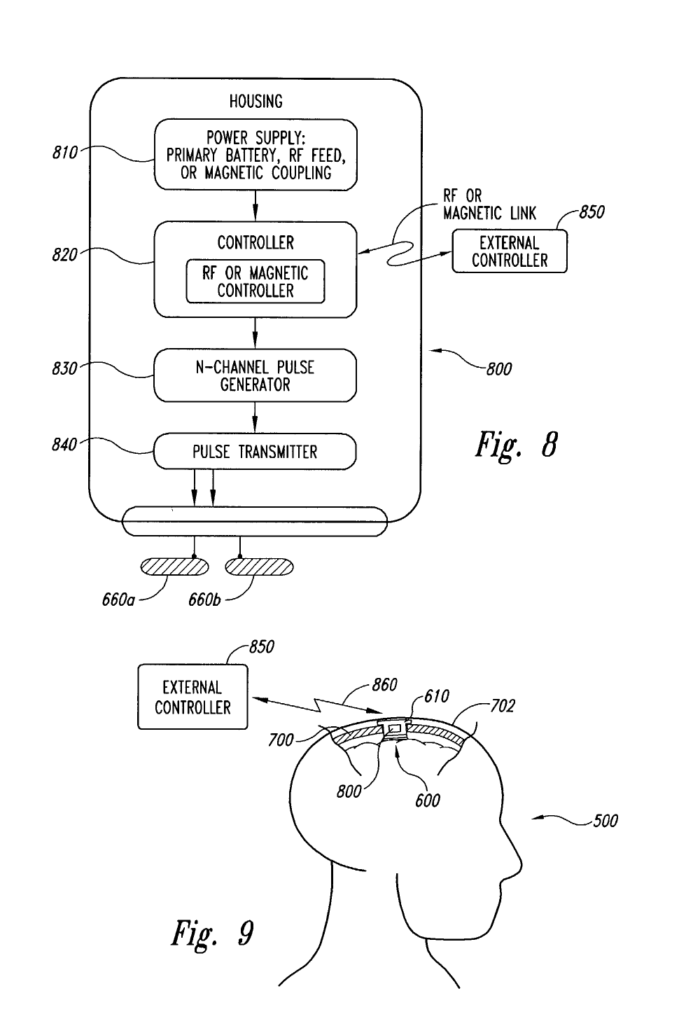

FIGS. 8 and 9 schematically illustrate an integrated pulse system 800 in accordance with one embodiment of the invention for being implanted in the cranium within the stimulation apparatus 600. Referring to FIG. 8, the pulse system 800 can include a power supply 810, an integrated controller 820, a pulse generator 830, and a pulse transmitter 840. The power supply 810 can be a primary battery, such as a rechargeable battery or another suitable device for storing electrical energy. In alternative embodiments, the power supply 810 can be an RF transducer or a magnetic transducer that receives broadcast energy emitted from an external power source and converts the broadcast energy into power for the electrical components of the pulse system 800. The integrated controller 820 can be a wireless device that responds to command signals sent by an external controller 850. The integrated controller 820, for example, can communicate with the external controller 850 by RF or magnetic links 860. The integrated controller 820 provides control signals to the pulse generator 830 in response to the command signals sent by the external controller 850. The pulse generator 830 can have a plurality of channels that send appropriate electrical pulses to the pulse transmitter 840, which is coupled to the electrodes 660. Suitable components for the power supply 810, the integrated controller 820, the pulse generator 830, and the pulse transmitter 840 are known to persons skilled in the art of implantable medical devices.

Referring to FIG. 9, the pulse system 800 can be carried by the support member 610 of the stimulation apparatus 600 in the manner described above with reference to FIGS. 6 and 7. The external controller 850 can be located externally to the patient 500 so that the external controller 850 can be used to control the pulse system 800. In one embodiment, several patients that require a common treatment can be simultaneously treated using a single external controller 850 by positioning the patients within the operating proximity of the controller 850. In an alternative embodiment, the external controller 850 can contain a plurality of operating codes and the integrated controller 820 for a particular patient can have an individual operating code. A single controller 850 can thus be used to treat a plurality of different patients by entering the appropriate operating code into the controller 850 corresponding to the particular operating codes of the integrated controllers 820 for the patients.

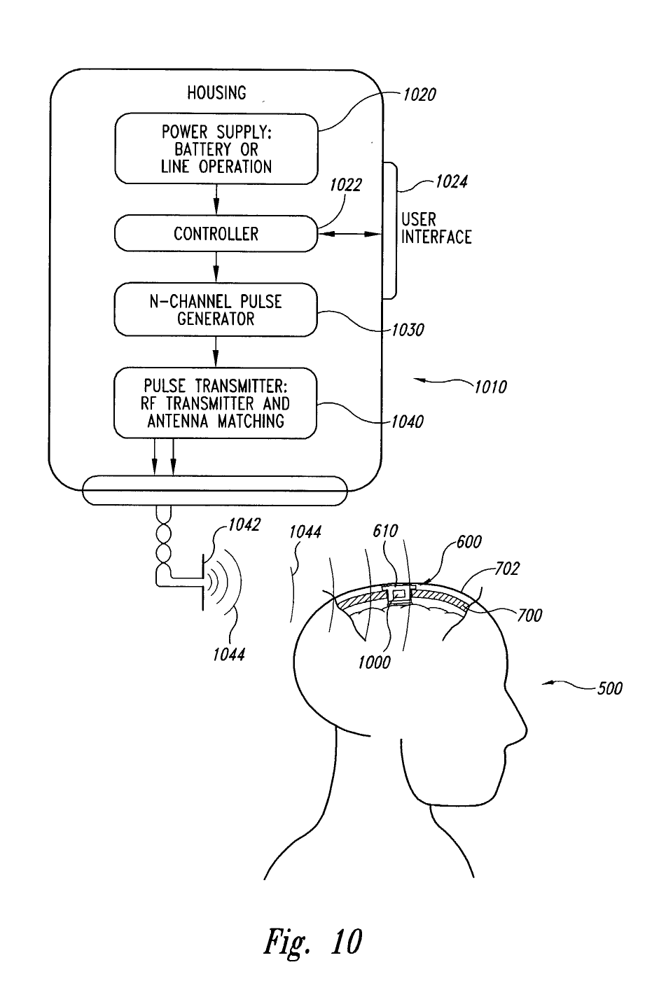

FIG. 10 is a schematic view illustrating a pulse system 1000 and an external controller 1010 for use with the stimulation apparatus 600 in accordance with another embodiment of the invention. In this embodiment, the external controller 1010 includes a power supply 1020, a controller 1022 coupled to the power supply 1020, and a user interface 1024 coupled to the controller 1022. The external controller 1010 can also include a pulse generator 1030 coupled to the power supply 1020, a pulse transmitter 1040 coupled to the pulse generator 1030, and an antenna 1042 coupled to the pulse transmitter 1040. The external controller 1010 generates the power and the pulse signal, and the antenna 1042 transmits a pulse signal 1044 to the pulse system 1000 in the stimulation apparatus 600. The pulse system 1000 receives the pulse signal 1044 and delivers an electrical pulse to the electrodes. The pulse system 1000, therefore, does not necessarily include an integrated power supply, controller and pulse generator within the housing 610 because these components are in the external controller 1010.

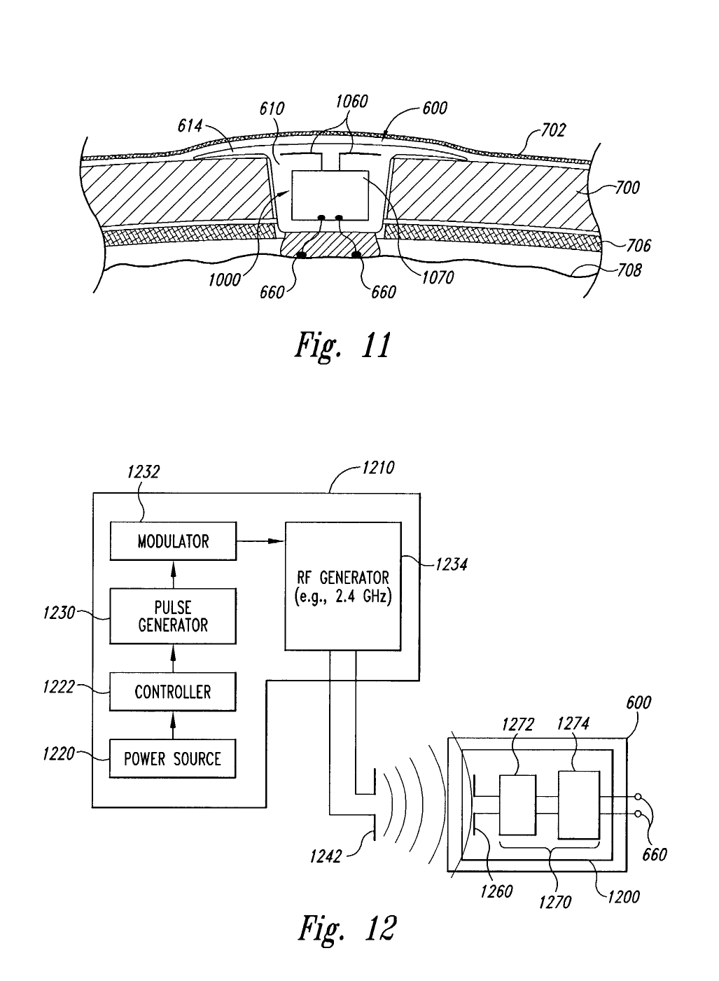

FIG. 11 is a schematic view illustrating an embodiment of the pulse system 1000 in greater detail. In this embodiment, the pulse system 1000 is carried by the support member 610 of the stimulation apparatus 600. The pulse system 1000 can include an antenna 1060 and a pulse delivery system 1070 coupled to the antenna 1060. The antenna 1060 receives the pulse signal 1044 from the external controller 1010 and sends the pulse signal 1044 to the pulse delivery system 1070, which transforms the pulse signal 1044 into electrical pulses. Accordingly, the electrodes 660 can be coupled to the pulse delivery system 1070. The pulse delivery system 1070 can include a filter to remove noise from the pulse signal 1044 and a pulse former that creates an electrical pulse from the pulse signal 1044. The pulse former can be driven by the energy in the pulse signal 1044, or in an alternative embodiment, the pulse system 1000 can also include an integrated power supply to drive the pulse former.

FIG. 12 is a schematic view illustrating an embodiment of pulse system 1200 for use in an embodiment of the stimulation apparatus 600, and an external controller 1210 for controlling the pulse system 1200 remotely from the patient using RF energy. In this embodiment, the external controller 1210 includes a power supply 1220, a controller 1222 coupled to the power supply 1220, and a pulse generator 1230 coupled to the controller 1222. The external controller 1210 can also include a modulator 1232 coupled to the pulse generator 1230 and an RF generator 1234 coupled to the modulator 1232. In operation, the external controller 1210 broadcasts pulses of RF energy via an antenna 1242.

The pulse system 1200 can be housed within the stimulation apparatus 600 (not shown). In one embodiment, the pulse system 1200 includes an antenna 1260 and a pulse delivery system 1270. The antenna 1260 incorporates a diode (not shown) that rectifies the broadcast RF energy from the antenna 1242. The pulse delivery system 1270 can include a filter 1272 and a pulse former 1274 that forms electrical pulses which correspond to the RF energy broadcast from the antenna 1242. The pulse system 1200 is accordingly powered by the RF energy in the pulse signal from the external controller 1210 such that the pulse system 1200 does not need a separate power supply carried by the stimulation apparatus 600.

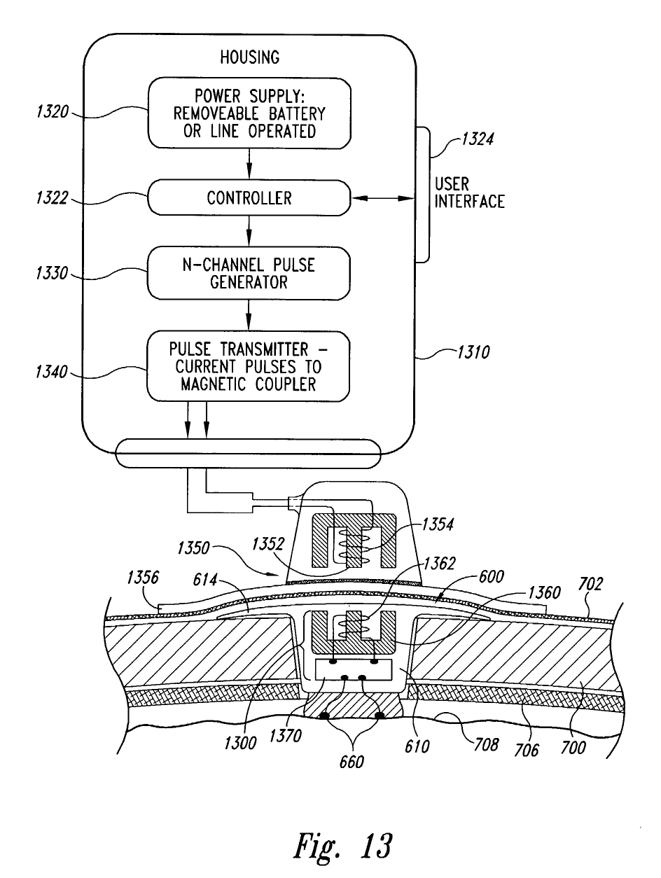

FIG. 13 is a cross-sectional view of a pulse system 1300 for use in another embodiment of the implantable stimulation apparatus 600, together with an external controller 1310 for remotely controlling the pulse system 1300 externally from the patient using magnetic energy. In this embodiment, the external controller 1310 includes a power supply 1320, a controller 1322 coupled to the power supply 1320, and a user interface 1324 coupled to the controller 1322. The external controller 1310 can also include a pulse generator 1330 coupled to the controller 1332, a pulse transmitter 1340 coupled to the pulse generator 1330, and a magnetic coupler 1350 coupled to the pulse transmitter 1340. The magnetic coupler 1350 can include a ferrite core 1352 and a coil 1354 wrapped around a portion of the ferrite core 1352. The coil 1354 can also be electrically connected to the pulse transmitter 1340 so that electrical pulses applied to the coil 1354 generate changes in a corresponding magnetic field. The magnetic coupler 1350 can also include a flexible cap 1356 to position the magnetic coupler 1350 over the implanted stimulation apparatus 600.

The pulse system 1300 can include a ferrite core 1360 and a coil 1362 wrapped around a portion of the ferrite core 1360. The pulse system 1310 can also include a pulse delivery system 1370 including a rectifier and a pulse former. In operation, the ferrite core 1360 and the coil 1362 convert the changes in the magnetic field generated by the magnetic coupler 1350 into electrical pulses that are sent to the pulse delivery system 1370. The electrodes 660 are coupled to the pulse delivery system 1370 so that electrical pulses corresponding to the electrical pulses generated by the pulse generator 1330 in the external controller 1310 are delivered to the stimulation site on the patient.

3. Electrode Configurations

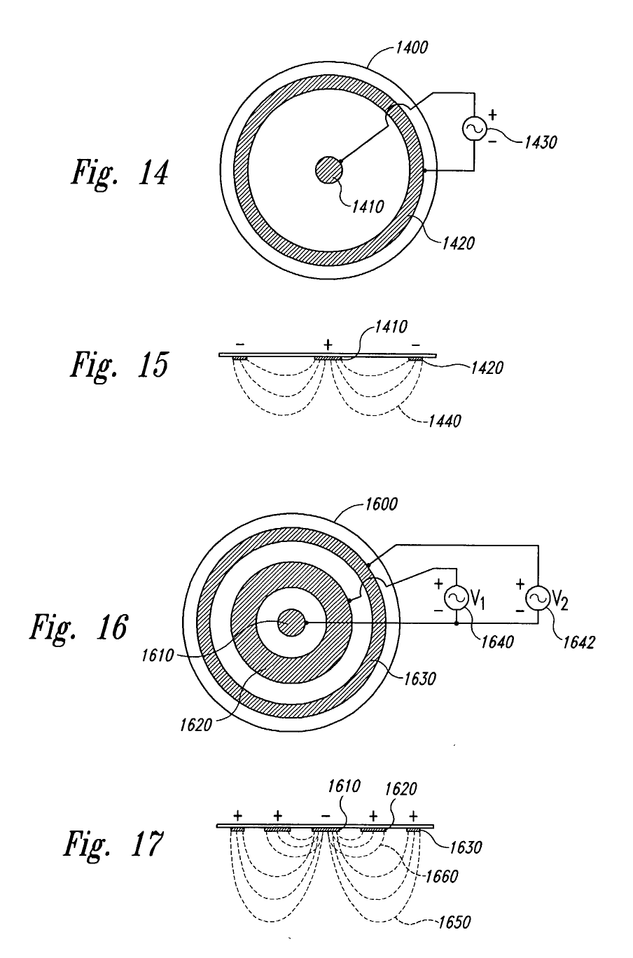

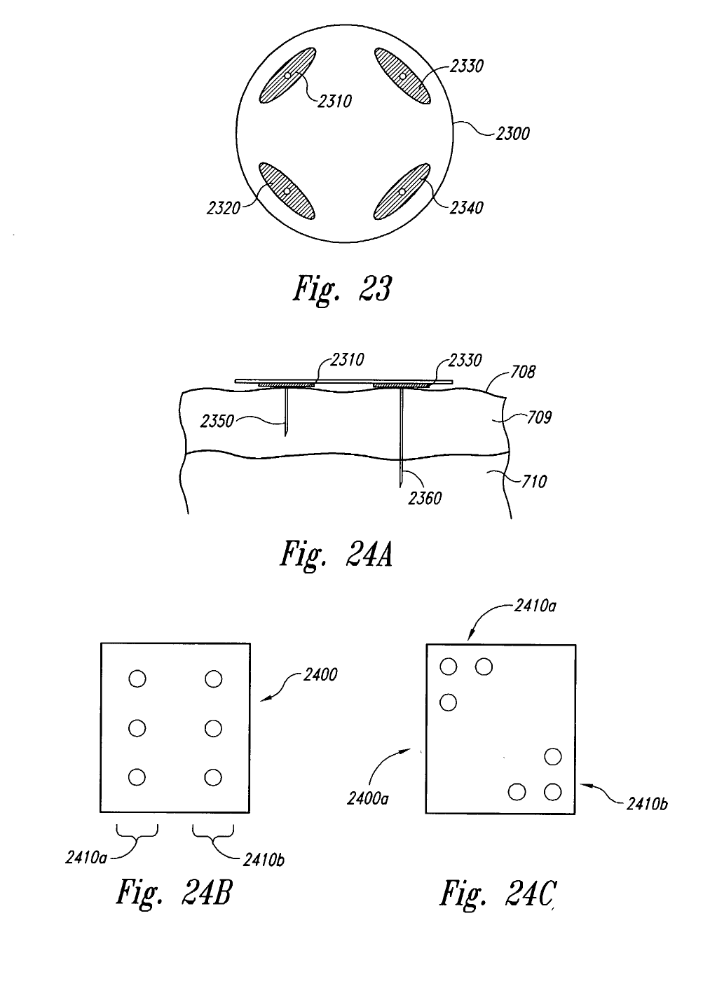

FIGS. 14-24c illustrate electrodes in accordance with various embodiments of the invention that can be used with the stimulation apparatus disclosed herein. FIGS. 14-22 illustrate embodiments of electrodes configured to apply an electrical current to a stimulation site at least proximate to the pial surface of the cortex, and FIGS. 23 and 24a illustrate embodiments of electrodes configured to apply an electrical current within the cortex or below the cortex. FIGS. 24b-c illustrate electrodes suitable for applying current at either location. It will be appreciated that other configurations of electrodes can also be used with other implantable stimulation apparatus.

FIG. 14 is a bottom plan view and FIG. 15 is a cross-sectional view of a stimulation apparatus 1400 in accordance with an embodiment of the invention. In this embodiment, the stimulation apparatus 1400 includes a first electrode 1410 and a second electrode 1420 concentrically surrounding the first electrode 1410. The first electrode 1410 can be coupled to the positive terminal of a pulse generator 1430, and the second electrode 1420 can be coupled to the negative terminal of the pulse generator 1430. Referring to FIG. 15, the first and second electrodes 1410 and 1420 generate a toroidal electric field 1440.

FIG. 16 is a bottom plan view and FIG. 17 is a cross-sectional view of a stimulation apparatus 1600 in accordance with another embodiment of the invention. In this embodiment, the stimulation apparatus 1600 includes a first electrode 1610, a second electrode 1620 surrounding the first electrode 1610, and a third electrode 1630 surrounding the second electrode 1620. The first electrode 1610 can be coupled to the negative terminals of a first pulse generator 1640 and a second pulse generator 1642; the second electrode 1620 can be coupled to the positive terminal of the first pulse generator 1640; and the third electrode 1630 can be coupled to the positive terminal of the second pulse generator 1642. In operation, the first electrode 1610 and the third electrode 1630 generate a first toroidal electric field 1650, and the first electrode the 1610 and the second electrode 1620 generate a second toroidal electric field 1660. The second toroidal electric field 1660 can be manipulated to vary the depth that the first toroidal electric field 1650 projects away from the base of the stimulation apparatus 1600.

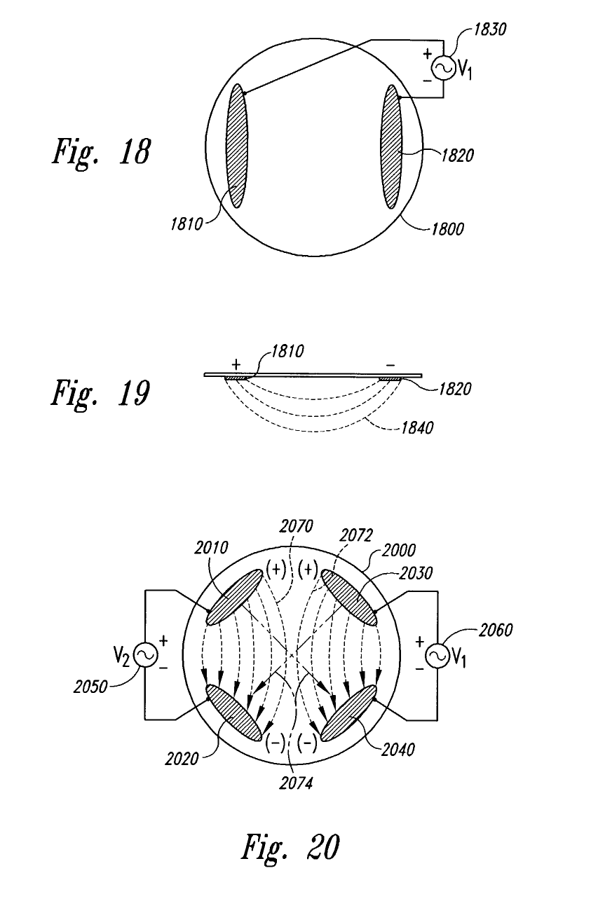

FIG. 18 is a bottom plan view and FIG. 19 is a cross-sectional view of a stimulation apparatus 1800 in accordance with yet another embodiment of the invention. In this embodiment, the stimulation apparatus 1800 includes a first electrode 1810 and a second electrode 1820 spaced apart from the first electrode 1810. The first and second electrodes 1810 and 1820 are linear electrodes which are coupled to opposite terminals of a pulse generator 1830. Referring to FIG. 19, the first and second electrodes 1810 and 1820 can generate an approximately linear electric field.

FIG. 20 is a bottom plan view of a stimulation apparatus 2000 in accordance with still another embodiment of the invention. In this embodiment, the stimulation apparatus 2000 includes a first electrode 2010, a second electrode 2020, a third electrode 2030, and a fourth electrode 2040. The first and second electrodes 2010 and 2020 are coupled to a first pulse generator 2050, and the third and fourth electrodes 2030 and 2040 are coupled to a second pulse generator 2060. More specifically, the first electrode 2010 is coupled to the positive terminal and the second electrode 2020 is coupled to the negative terminal of the first pulse generator 2050, and the third electrode 2030 is coupled to the positive terminal and the fourth electrode 2040 is coupled to the negative terminal of the second pulse generator 2060. The first and second electrodes 2010 and 2020 are expected to generate a first electric field 2070, and the third and fourth electrodes 2030 and 2040 are expected to generate a second electric field 2072. It will be appreciated that the ions will be relatively free to move through the brain such that a number of ions will cross between the first and second electric fields 2070 and 2072 as shown by arrows 2074. This embodiment provides control of electric field gradients at the stimulation sites.

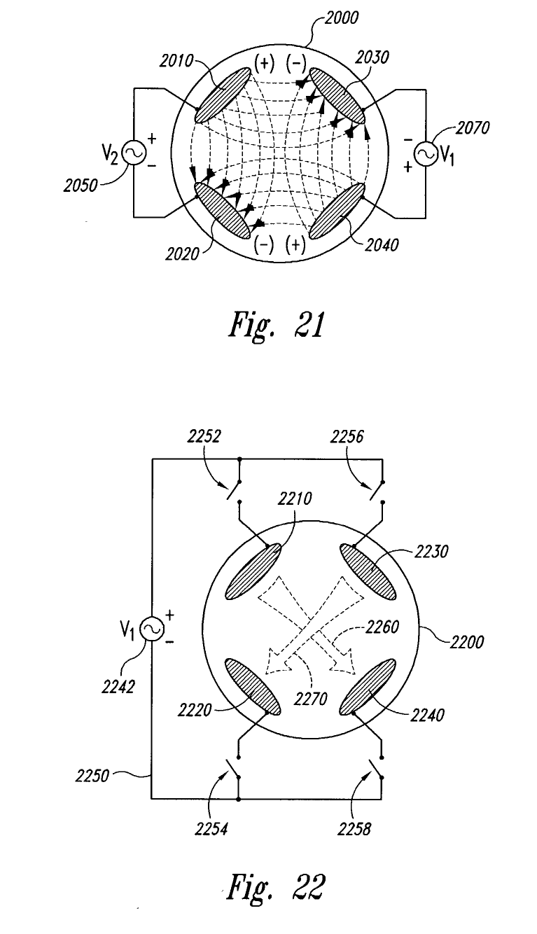

FIG. 21 is a bottom plan view of another embodiment of the stimulation apparatus 2000. In this embodiment, the first electrode 2010 is coupled to the positive terminal and the second electrode 2020 is coupled to the negative terminal of the first pulse generator 2050. In contrast to the embodiment shown in FIG. 20, the third electrode 2030 is coupled to the negative terminal and the fourth electrode 2040 is coupled to the positive terminal of the second pulse generator 2070. It is expected that this electrode arrangement will result in a plurality of electric fields between the electrodes. This allows control of the direction or orientation of the electric field.

FIG. 22 is a bottom plan view that schematically illustrates a stimulation apparatus 2200 in accordance with still another embodiment of the invention. In this embodiment, the stimulation apparatus 2200 includes a first electrode 2210, a second electrode 2220, a third electrode 2230, and a fourth electrode 2240. The electrodes are coupled to a pulse generator 2242 by a switch circuit 2250. The switch circuit 2250 can include a first switch 2252 coupled to the first electrode 2210, a second switch 2254 coupled to the second electrode 2220, a third switch 2256 coupled to the third electrode 2230, and a fourth switch 2258 coupled to the fourth electrode 2240. In operation, the switches 2252-2258 can be opened and closed to establish various electric fields between the electrodes 2210-2240. For example, the first switch 2252 and the fourth switch 2258 can be closed in coordination with a pulse from the pulse generator 2242 to generate a first electric field 2260, and/or the second switch 2254 and the third switch 2256 can be closed in coordination with another pulse from the pulse generator 2242 to generate a second electric field 2270. The first and second electric fields 2260 and 2270 can be generated at the same pulse to produce concurrent fields or alternating pulses to produce alternating or rotating fields.

FIG. 23 is a bottom plan view and FIG. 24a is a side elevational view of a stimulation apparatus 2300 in accordance with another embodiment of the invention. In this embodiment, the stimulation apparatus 2300 has a first electrode 2310, a second electrode 2320, a third electrode 2330, and a fourth electrode 2340. The electrodes 2310-2340 can be configured in any of the arrangements set forth above with reference to FIGS. 14-22. The electrodes 2310-2340 also include electrically conductive pins 2350 and/or 2360. The pins 2350 and 2360 can be configured to extend below the pial surface of the cortex. For example, because the length of the pin 2350 is less than the thickness of the cortex 709, the tip of the pin 2350 will accordingly conduct the electrical pulses to a stimulation site within the cortex 709 below the pial surface. The length of the pin 2360 is greater than the thickness of the cortex 709 to conduct the electrical pulses to a portion of the brain below the cortex 709, such as a deep brain region 710. The lengths of the pins are selected to conduct the electrical pulses to stimulation sites below the pia mater 708. As such, the length of the pins 2350 and 2360 can be the same for each electrode or different for individual electrodes. Additionally, only a selected portion of the electrodes and the pins can have an exposed conductive area. For example, the electrodes 2310-2340 and a portion of the pins 2350 and 2360 can be covered with a dielectric material so that only exposed conductive material is at the tips of the pins. It will also be appreciated that the configurations of electrodes set forth in FIGS. 14-22 can be adapted to apply an electrical current to stimulation sites below the pia mater by providing pin-like electrodes in a matter similar to the electrodes shown in FIGS. 23 and 24a.

In other embodiments, apparatuses suitable for implantation below or above the pial surface can have other embodiments. For example, as shown in FIG. 24b, an apparatus 2400a can include two sets of electrodes 2410 (shown as a first set 2410a and a second set 2410b) arranged in opposing rows. Each set of electrodes can be coupled to opposite poles of a power source. In another embodiment (shown in FIG. 24c), an apparatus 2400b can include two sets of electrodes 2410a, 2410b, arranged in opposing corners of the apparatus 2400b and coupled to opposite poles of a power source. In other embodiments, the apparatus and electrodes can have other suitable arrangements.

Several embodiments of the stimulation apparatus described above with reference to FIGS. 6-24c are expected to be more effective than existing transcranial or subcranial stimulation devices. In addition to positioning the electrodes under the skull, many embodiments of the stimulation apparatus described above also accurately focus the electrical energy in desired patterns relative to the pia mater 708, the dura mater 706, and/or the cortex 709. It will be appreciated that transcranial devices may not accurately focus the energy because the electrodes or other types of energy emitters are positioned relatively far from the stimulation sites and the skull diffuses some of the energy. Also, existing subcranial devices generally merely place the electrodes proximate to a specific nerve, but they do not provide electrode configurations that generate an electrical field in a pattern designed for the stimulation site. Several of the embodiments of the stimulation apparatus described above with reference to FIGS. 6-24c overcome this drawback because the electrodes can be placed against the neurons at the desired stimulation site. Additionally, the electrode configurations of the stimulation apparatus can be configured to provide a desired electric field that is not diffused by the skull 700. Therefore, several embodiments of the stimulation apparatus in accordance with the invention are expected to be more effective because they can accurately focus the energy at the stimulation site.

4. Implantable Stimulation Apparatus With Biasing Elements

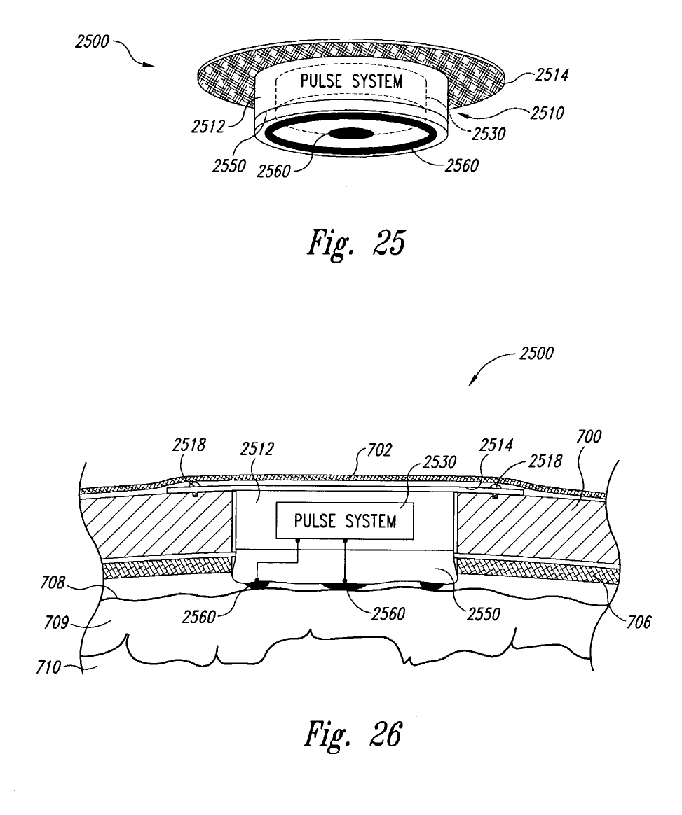

FIGS. 25-30 illustrate several embodiments of stimulation apparatus having a biasing element in accordance with a different aspect of the invention. The stimulation apparatus shown in FIGS. 25-30 can be similar to those described above with reference to FIGS. 6-24c. Therefore, the embodiments of the stimulation apparatus shown in FIGS. 25-30 can have the same pulse systems, support members and electrode configurations described above with reference to FIGS. 6-24c.

FIG. 25 is an isometric view and FIG. 26 is a cross-sectional view of a stimulation apparatus 2500 in accordance with an embodiment of the invention. In one embodiment, the stimulation apparatus 2500 includes a support member 2510, a pulse-system 2530 carried by the support member 2510, and first and second electrodes 2560 coupled to the pulse system 2530. The support member 2510 can be identical or similar to the support member 610 described above with reference to FIGS. 6 and 7. The support member 2510 can accordingly include a housing 2512 configured to be implanted in the skull 700 and an attachment element 2514 configured to be connected to the skull 700 by fasteners 2518 (FIG. 2), an adhesive, and/or an anchor. The pulse system 2530 can be identical or similar to any of the pulse systems described above with reference to FIGS. 6-13, and the first and second electrodes 2560 can have any of the electrode configurations explained above with reference to FIGS. 14-24c. Unlike the stimulation apparatus described above, however, the stimulation apparatus 2500 includes a biasing element 2550 coupled to the electrodes 2560 to mechanically bias the electrodes 2560 away from the support member 2510. In an alternative embodiment, the biasing element 2550 can be positioned between the housing 2512 and the attachment element 2514, and the electrodes 2560 can be attached directly to the housing 2512. As explained in more detail below, the biasing element 2550 can be a compressible member, a fluid filled bladder, a spring, or any other suitable element that resiliently and/or elastically drives the electrodes 2560 away from the support member 2510.

FIG. 26 illustrates an embodiment of the stimulation apparatus 2500 after it has been implanted into the skull 700 of a patient. When the fasteners 2518 are attached to the skull 700, the biasing element 2550 should be compressed slightly so that the electrodes 2560 contact the stimulation site. In the embodiment shown in FIG. 26, the compressed biasing element 2550 gently presses the electrodes 2560 against the surface of the pia mater 708. In another embodiment (for example, when the apparatus 2500 does not extend through the dura mater 706), the biasing element 2550 can gently press the electrodes 2560 against the surface of the dura mater 706 . It is expected that the biasing element 2550 will provide a uniform, consistent contact between the electrodes 2560 and the pial surface (or dural surface) of the cortex 709. The stimulation apparatus 2500 is expected to be particularly useful when the implantable device is attached to the skull and the stimulation site is on the pia mater 708 or the dura mater 706. It can be difficult to position the contacts against the pia mater 708 because the distance between the skull 700, the dura mater 706, and the pia mater 708 varies within the cranium as the brain moves relative to the skull, and also as the depth varies from one patient to another. The stimulation apparatus 2500 with the biasing element 2550 compensates for the different distances between the skull 700 and the pia mater 708 (or the dura mater 706) so that a single type of device can inherently fit several different patients. Moreover, the stimulation apparatus 2500 with the biasing element 2550 adapts to changes as the brain moves within the skull. In contrast to the stimulation apparatus 2500 with the biasing element 2550, an implantable device that does not have a biasing element 2550 may not fit a particular patient or may not consistently provide electrical contact to the pia mater or dura mater.

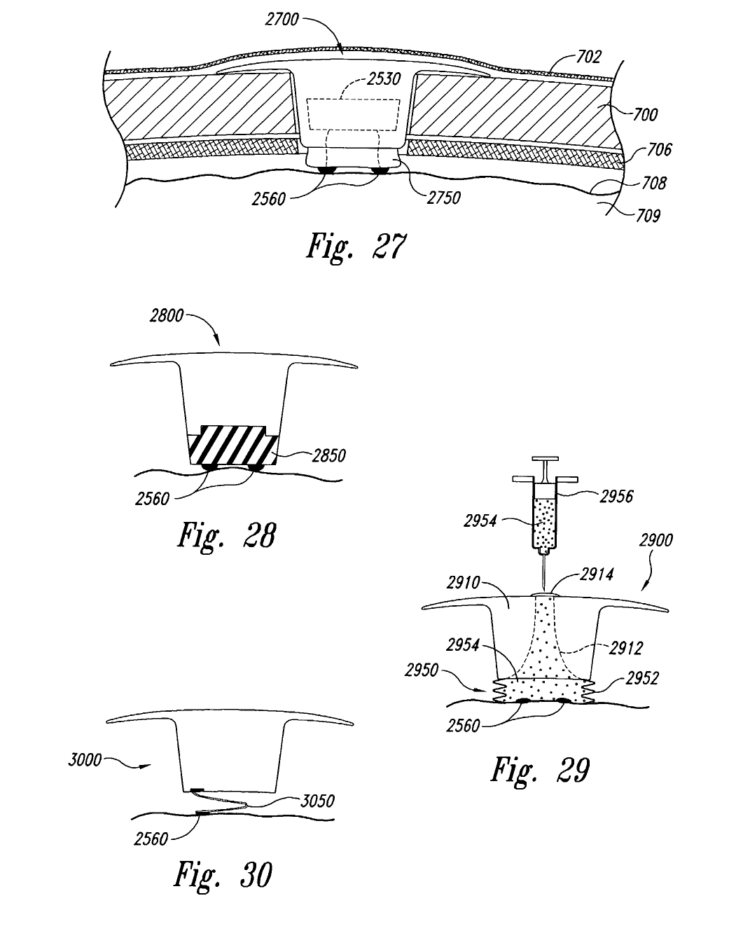

FIGS. 27 and 28 are cross-sectional views of stimulation apparatus in which the biasing elements are compressible members. FIG. 27, more specifically, illustrates a stimulation apparatus 2700 having a biasing element 2750 in accordance with an embodiment of the invention. The stimulation apparatus 2700 can have an integrated pulse system 2530 and electrodes 2560 coupled to the pulse system 2530 in a manner similar to the stimulation apparatus 2500. The biasing element 2750 in this embodiment is a compressible foam, such as a biocompatible closed cell foam or open cell foam. As best shown in FIG. 27, the biasing element 2750 compresses when the stimulation apparatus 2700 is attached to the skull. FIG. 28 illustrates a stimulation apparatus 2800 having a biasing element 2850 in accordance with another embodiment of the invention. The biasing element 2850 can be a compressible solid, such as silicon rubber or other suitable compressible materials. The electrodes 2560 are attached to the biasing element 2850.

FIG. 29 is a cross-sectional view of a stimulation apparatus 2900 having a biasing element 2950 in accordance with another embodiment of the invention. The stimulation apparatus 2900 can have a support member 2910 including an internal passageway 2912 and a diaphragm 2914. The biasing element 2950 can include a flexible bladder 2952 attached to the support member 2910, and the electrodes 2560 can be attached to the flexible bladder 2952. In operation, the flexible bladder 2952 is filled with a fluid 2954 until the electrodes 2560 press against the stimulation site. In one embodiment, the flexible bladder 2952 is filled by inserting a needle of a syringe 2956 through the diaphragm 2914 and injecting the fluid 2954 into the internal passageway 2912 and the flexible bladder.

FIG. 30 is a cross-sectional view of a stimulation apparatus 3000 having a biasing element 3050 in accordance with another embodiment of the invention. In this embodiment, the biasing element 3050 is a spring and the electrodes 2560 are attached to the spring. The biasing element 3050 can be a wave spring, a leaf spring, or any other suitable spring that can mechanically bias the electrodes 2560 against the stimulation site.

Although several embodiments of the stimulation apparatus shown in FIGS. 25-30 can have a biasing element and any of the pulse systems set forth above with respect to FIGS. 6-13, it is not necessary to have a pulse system contained within the support member. Therefore, certain embodiments of implantable stimulation apparatus in accordance with the invention can have a pulse system and/or a biasing member in any combination of the embodiments set forth above with respect to FIGS. 6-30.

5. Implantable Stimulation Apparatus With External Pulse Systems

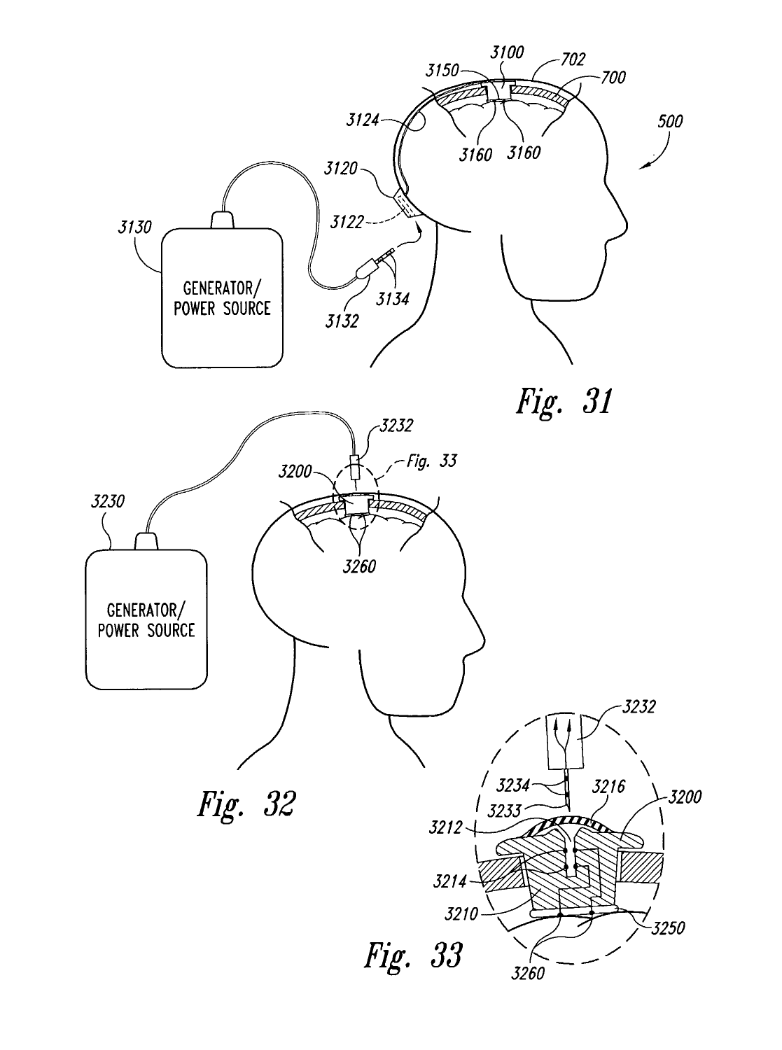

FIGS. 31-35 are schematic cross-sectional views of various embodiments of implantable stimulation apparatus having external pulse systems. FIG. 31, more specifically, illustrates an embodiment of a stimulation apparatus 3100 having a biasing element 3150 to which a plurality of electrodes 3160 are attached in a manner similar to the stimulation apparatus described above with reference to FIGS. 25-30. It will be appreciated that the stimulation apparatus 3100 may not include the biasing element 3150. The stimulation apparatus 3100 can also include an external receptacle 3120 having an electrical socket 3122 and an implanted lead line 3124 coupling the electrodes 3160 to contacts (not shown) in the socket 3122. The lead line 3124 can be implanted in a subcutaneous tunnel or other passageway in a manner known to a person skilled and art.

The stimulation apparatus 3100, however, does not have an internal pulse system carried by the portion of the device that is implanted in the skull 700 of the patient 500. The stimulation apparatus 3100 receives electrical pulses from an external pulse system 3130. The external pulse system 3130 can have an electrical connector 3132 with a plurality of contacts 3134 configured to engage the contacts within the receptacle 3120. The external pulse system 3130 can also have a power supply, controller, pulse generator, and pulse transmitter to generate the electrical pulses. In operation, the external pulse system 3130 sends electrical pulses to the stimulation apparatus 3100 via the connector 3132, the receptacle 3120, and the lead line 3124.

FIGS. 32 and 33 illustrate an embodiment of a stimulation apparatus 3200 for use with an external pulse system in accordance with another embodiment of the invention. Referring to FIG. 33, the stimulation apparatus 3200 can include a support structure 3210 having a socket 3212, a plurality of contacts 3214 arranged in the socket 3212, and a diaphragm 3216 covering the socket 3212. The stimulation apparatus 3200 can also include a biasing element 3250 and a plurality of electrodes 3260 attached to the biasing element 3250. Each electrode 3260 is directly coupled to one of the contacts 3214 within the support structure 3210. It will be appreciated that an alternative embodiment of the stimulation apparatus 3200 does not include the biasing element 3250.

Referring to FIGS. 32 and 33 together, the stimulation apparatus 3200 receives the electrical pulses from an external pulse system 3230 that has a power supply, controller, pulse generator, and pulse transmitter. The external pulse system 3230 can also include a plug 3232 having a needle 3233 (FIG. 33) and a plurality of contacts 3234 (FIG. 33) arranged on the needle 3233 to contact the internal contacts 3214 in the socket 3212. In operation, the needle 3233 is inserted into the socket 3212 to engage the contacts 3234 with the contacts 3214, and then the pulse system 3230 is activated to transmit electrical pulses to the electrodes 3260.

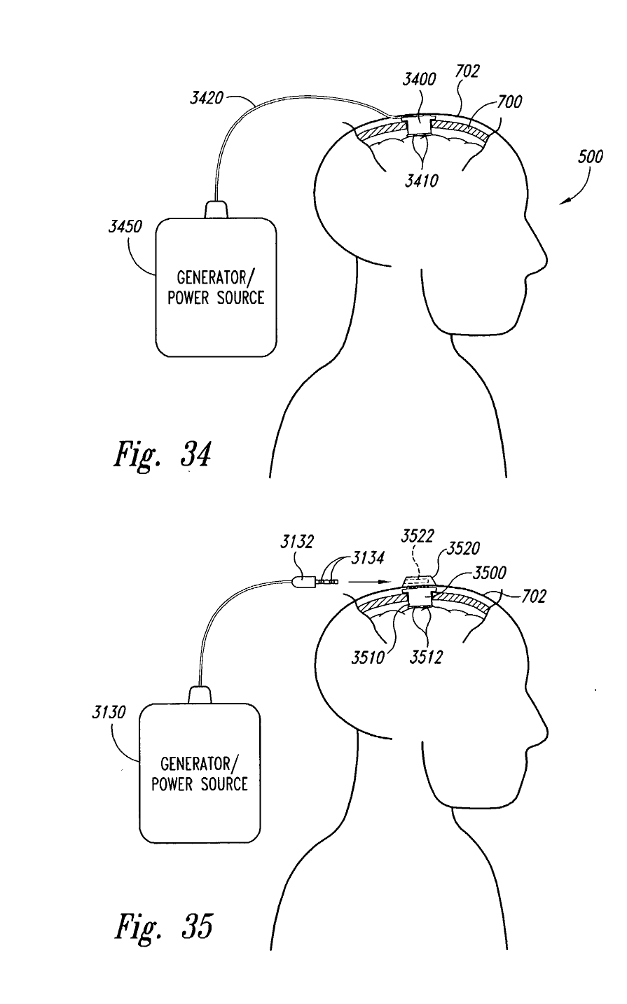

FIGS. 34 and 35 illustrate additional embodiments of stimulation apparatus for use with external pulse systems. FIG. 34 illustrates an embodiment of a stimulation apparatus 3400 having electrodes 3410 coupled to a lead line 3420 that extends under the scalp 702 of the patient 500. The lead line 3420 is coupled to an external pulse system 3450. FIG. 35 illustrates an embodiment of a stimulation apparatus 3500 having a support member 3510, electrodes 3512 coupled to the support member 3510, and an external receptacle 3520 mounted on the scalp 702. The external receptacle 3520 can also be connected to the support member 3510. The external receptacle 3520 can have a socket 3522 with contacts (not shown) electrically coupled to the electrodes 3512. The stimulation apparatus 3500 can be used with the external pulse system 3130 described above with reference to FIG. 31 by inserting the plug 3132 into the socket 3522 until the contacts 3134 on the plug 3132 engage the contacts within the socket 3522.

6. Alternate Embodiments of Implantable Stimulation Apparatus

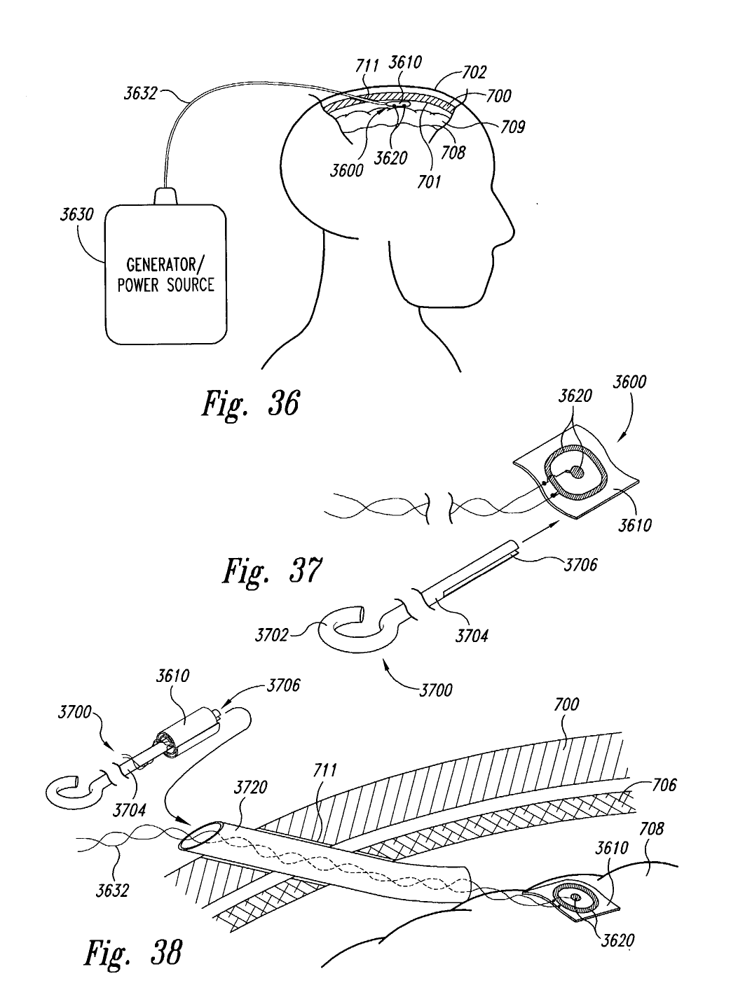

FIG. 36 is a schematic cross-sectional view of an implantable stimulation apparatus 3600 in accordance with another embodiment of the invention. In one embodiment, the stimulation apparatus 3600 has a support structure 3610 and a plurality of electrodes 3620 coupled to the support structure 3610. The support structure 3610 can be configured to be implanted under the skull 700 between an interior surface 701 of the skull 700 and the pial or dural surface of the brain. The support structure 3610 can be a flexible or compressible body such that the electrodes 3620 contact the pia mater 708 or the dura mater 706 when the stimulation apparatus 3600 is implanted under the skull 700. In other embodiments, the support structure 3610 can position the electrodes 3620 so that they are proximate to, but not touching, the pia mater 708 or the dura matter 706.

In one embodiment, the stimulation apparatus 3600 can receive electrical pulses from an external controller 3630. For example, the external controller 3630 can be electrically coupled to the stimulation apparatus 3600 by a lead line 3632 that passes through a hole 711 in the skull 700. In an alternative embodiment, the stimulation apparatus 3600 can include an integrated pulse system similar to the pulse systems described above with reference to FIGS. 6-13. Such an embodiment of the stimulation apparatus 3600 can accordingly use a wireless external control unit. It will be appreciated that the electrodes 3620 of the stimulation apparatus 3600 can have several of the electrode configurations described above with reference to FIGS. 14-24c.

FIGS. 37 and 38 illustrate one embodiment of the implantable stimulation apparatus 3600. Referring to FIG. 37, the support structure 3610 can be a flexible substrate and the electrodes 3620 can be conductive elements that are printed onto the flexible substrate. The stimulation apparatus 3600, for example, can be manufactured in a manner similar to flexible printed circuit assemblies that are used in electrical components. The stimulation apparatus 3600 can be implanted under the skull 700 using an insertion tool 3700. In one embodiment, the insertion tool 3700 has a handle 3702 and a shaft 3704 projecting from the handle 3702. The shaft 3704 can have a slot 3706 configured to receive a flat portion of the support member 3610. Referring to FIG. 38, the support member 3610 is wrapped around the shaft 3704, and then the stimulation apparatus 3600 is passed to a tube 3720 positioned in the hole 711 through the scalp 700 and the dura mater 706. After the stimulation apparatus 3600 has been passed through the tube 3720, it is unfurled to place the electrodes 3620 at least proximate to the pia mater 708 or the dura mater 706. The electrodes 3620 can be coupled to an external controller by the lead lines 3632.

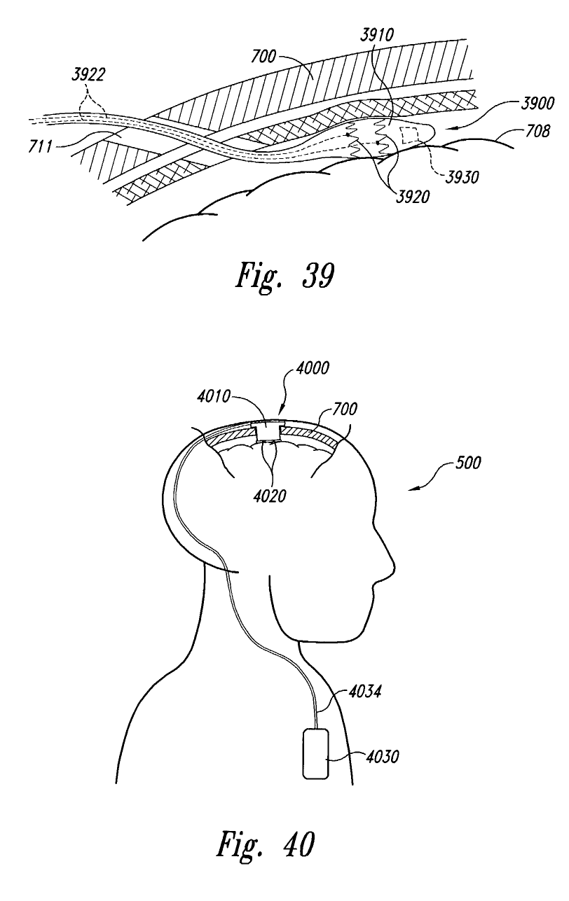

FIG. 39 illustrates another embodiment of an implantable stimulation apparatus 3900 that is also configured to be positioned between the skull 700 and the pia mater 708. In one embodiment, the stimulation apparatus 3900 can include a support member 3910 and a plurality of electrodes 3920 coupled to the support member 3910. The electrodes 3920 can be coupled to individual lead lines 3922 to connect the electrodes 3920 to an external pulse system. In an alternative embodiment, an integrated pulse system 3930 can be carried by the support member 3910 so that the electrodes 3920 can be coupled directly to the integrated pulse system 3930 without external lead lines 3922. The support member 3910 can be a resiliently compressible member, an inflatable balloon-like device, or a substantially solid incompressible body. In the particular embodiment shown in FIG. 39, the support member 3910 is an inflatable balloon-like device that carries the electrodes 3920. In operation, the stimulation apparatus 3900 is implanted by passing the distal end of the support member 3910 through the hole 711 in the skull 700 until the electrodes 3920 are positioned at a desired stimulation site.

FIG. 40 is a schematic illustration of a stimulation apparatus 4000 together with an internal pulse system 4030 in accordance with another embodiment of the invention. The stimulation apparatus 4000 can include a support member 4010, a biasing element 4015 carried by the support member 4010, and a plurality of electrodes 4020 carried by the biasing element 4015. The internal pulse system 4030 can be similar to any of the integrated pulse systems described above with reference to FIGS. 6-13, but the internal pulse system 4030 is not an integrated pulse system because it is not carried by the housing 4010. The internal pulse system 4030 can be coupled to the electrodes 4020 by a cable 4034. In a typical application, the cable 4034 is implanted subcutaneously in a tunnel from a subclavicular region, along the back of the neck, and around the skull. The stimulation apparatus 4000 can also include any of the electrode configurations described above with reference to FIGS. 14-24.

D. DEVICES FOR ELECTRICAL STIMULATION OF CELLS IMPLANTED IN THE NERVOUS SYSTEM

FIGS. 41A-B schematically illustrate a procedure for implanting cells in the brain in accordance with an embodiment of the invention. Referring to FIG. 41A, an implantation site 502 is identified, for example, in accordance with an embodiment of the diagnostic procedure 102 described above with reference to FIG. 1C. Alternatively, the implantation site 502 can be selected in accordance with other techniques. In either embodiment, the implantation process can include removing from the patient 500 a skull section 504 covering the implantation site 502. A syringe 4102 containing the cells suspended in a solution can be used to implant the cells at the implantation site 502. Alternatively, other techniques can be used to implant cells at the implantation site 502.

Referring now to FIG. 41B, an implantable stimulation apparatus 510 can be implanted in the patient 500 at least proximate to the location at which the cells have been implanted. In one aspect of this embodiment, the apparatus 510 can be implanted after the cells have been implanted, and in an alternative embodiment, the apparatus 510 can be implanted before the cells. In either embodiment, the apparatus 510 can transmit electrical current through the tissue surrounding the implanted cells. One feature of an embodiment of the foregoing arrangement is that the cells can be implanted directly into the surrounding native tissue and stimulated via the surrounding tissue. Accordingly, this arrangement does not require an implanted substrate to support the cells and transmit electrical signals to the cells. An advantage of this approach when compared with some conventional arrangements (such as that disclosed by Shastri in U.S. Pat. No. 6,095,148) is that it can reduce the complexity of the device and the amount of non-native material that must be implanted in the patient.

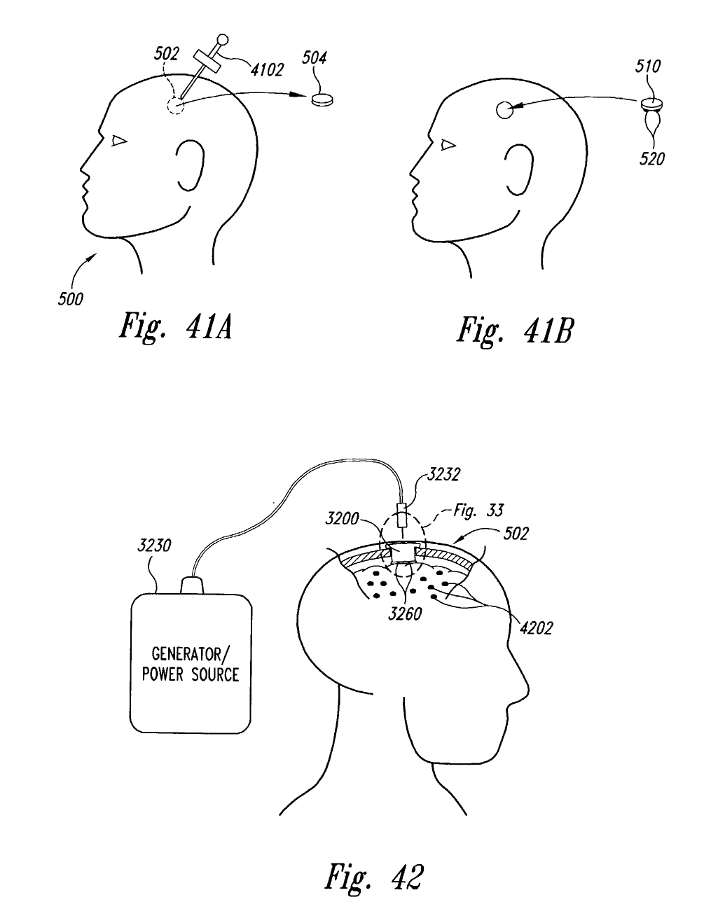

FIG. 42 schematically illustrates a procedure for electrically stimulating cells implanted in the brain in accordance with an embodiment of the invention. In one aspect of this embodiment, a plurality of electrodes 3260 are positioned at least proximate to the implantation site 502 to direct an electrical current to implanted cells 4202 via the tissue surrounding the implanted cells 4202. In one aspect of this embodiment, stimulation can be delivered to the electrodes 3260 proximate to the implanted cells 4202 by a pulse generator 3230 positioned external to the body. Alternatively, the pulse generator can be implanted, for example, in a manner generally similar to that described above with reference to any of FIGS. 6-30.

FIG. 43 schematically illustrates a procedure for electrically stimulating cells 4308 implanted in or proximate to the spinal cord 4302 in accordance with another embodiment of the invention. The stimulation apparatus 4304 can include leads 4306 and electrodes 4307 that are epidural, intrathecal, or placed into the spinal cord itself. The stimulation apparatus 4304 used in accordance with this procedure can be one of several commercially available neurostimulators, such as those manufactured by Medtronic of Minneapolis, Minn., or one of several devices disclosed in U.S. Pat. No. 6,058,331 to King, incorporated herein in its entirety by reference. These devices may also be used to stimulate cells implanted in a peripheral nerve of a patient, such as the peripheral nerves 4310 identified in FIG. 43 as cervical nerves, thoracic nerves, lumbar nerves, sacral nerves and coccygeal nerves. Alternatively, any of the foregoing devices can be used to stimulate other peripheral nerves.

FIG. 44 schematically illustrates a procedure for electrically stimulating cells 4410 in accordance with another embodiment of the invention. In one aspect of this embodiment, cells 4410 are grown in a conductive medium 4406. A pulse generator 4402 can deliver an electrical current in vitro to electrodes 4404 (or electrode plates) implanted in the conductive medium 4406 to electrically stimulate the cells 4410. After the cells 4410 have been electrically stimulated for a selected period of time, they can be removed from the conductive medium 4406 and implanted in the patient, as described above with reference to FIG. 5E. For example, the cells 4410 can be stimulated in vitro until they begin to exhibit characteristics of action potential cells. As described above, the electrical stimulation can be continued after implantation with the same or a different current level and/or modulation.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

1. A method of cell therapy, comprising:
preparing at least partially undifferentiated cells for implantation;
implanting the at least partially undifferentiated cells at an implantation site of a nervous system of a patient;
positioning at least one electrode in communication with the implantation site of the nervous system of the patient; and
differentiating the at least partially undifferentiated cells into cells with increased neural characteristics when compared to the at least partially undifferentiated cells by applying an electrical potential to the at least one electrode while the electrode is in communication with the implantation site of the nervous system.

2. The method of claim 1 wherein positioning at least one electrode includes positioning at least one electrode at least proximate to the implantation site.

3. The method of claim 1 wherein positioning at least one electrode includes positioning at least one electrode proximate to a native cell and communicating with the at least partially undifferentiated cells via the native cell.

4. The method of claim 1 wherein preparing the at least partially undifferentiated cells includes applying an electrical stimulation to the at least partially undifferentiated cells while the at least partially undifferentiated cells are external to a patient.

5. The method of claim 1 wherein the at least partially undifferentiated cells are selected to include stem cells, precursor cells, and/or progenitor cells.

6. The method of claim 1 wherein implanting the at least partially undifferentiated cells at an implantation site of a nervous system of a patient includes implanting the undifferentiated cells directly into the patient's tissue without a substrate and wherein applying an electrical potential includes directing an electrical current from the at least one electrode through the tissue adjacent to the at least partially undifferentiated cells and to the at least partially undifferentiated cells.

7. The method of claim 1 wherein implanting the at least partially undifferentiated cells at an implantation site of a nervous system of a patient includes implanting the at least partially undifferentiated cells at an implantation site of a spinal cord of a patient.

8. The method of claim 1 wherein implanting the at least partially undifferentiated cells at an implantation site of a nervous system of a patient includes implanting the at least partially undifferentiated cells at an implantation site of a brain of the patient.

9. The method of claim 1 wherein implanting the at least partially undifferentiated cells at an implantation site of a nervous system of a patient includes implanting the at least partially undifferentiated cells at an implantation site of a peripheral nerve of the patient.

10. The method of claim 1 wherein the at least one electrode includes a first electrode, and wherein the method further comprises positioning a second electrode at least proximate to the implantation site of the nervous system of the patient, and wherein applying an electrical potential includes applying a voltage of from about ±1 mV to about ±10 V between the first electrode and the second electrode while the electrodes are at least proximate to the implantation site of the nervous system.

11. The method of claim 1 wherein applying an electrical potential includes generating electrical pulses at a rate of from about 1 to about 1000 Hz.

12. The method of claim 1 wherein differentiating the at least partially undifferentiated cells into cells with increased neural characteristics includes applying an electrical potential to the at least one electrode at a first voltage until the at least partially undifferentiated cells develop action potentials and then applying an electrical potential to the at least one electrode at a second voltage less than the first voltage after the at least partially undifferentiated cells develop action potentials.

13. The method of claim 1 wherein differentiating the at least partially undifferentiated cells into cells with increased neural characteristics includes ceasing to apply an electrical potential to the at least one electrode after the at least partially undifferentiated cells develop increased action potentials.

14. The method of claim 1, further comprising ascertaining a threshold for generating action potentials for the at least partially undifferentiated cells at the implantation site of the nervous system, and wherein applying an electrical potential includes placing a subthreshold voltage less than the threshold for generating action potentials.

15. The method of claim 1 wherein the at least one electrode includes a first electrode, and wherein the method further comprises:
ascertaining a threshold for generating action potentials for the at least partially undifferentiated cells at the implantation site of the nervous system; and
positioning a second electrode at least proximate to the implantation site of the nervous system, and wherein applying an electrical potential includes placing a subthreshold voltage between the first electrode and the second electrode, wherein the subthreshold voltage is approximately 10% to approximately 50% less than the threshold for generating an action potential.

16. The method of claim 1 wherein the at least one electrode includes a first electrode, and wherein the method further comprises:
ascertaining a threshold for generating electrophysiologic signals associated with a neural function; and
positioning a second electrode at least proximate to the implantation site of the nervous system, and wherein applying an electrical potential includes placing a subthreshold voltage between the first electrode and the second electrode, wherein the subthreshold voltage is less than the threshold for generating electrophysiologic signals.

17. The method of claim 1 wherein the at least one electrode includes a first electrode, and wherein the method further comprises:
ascertaining a threshold for generating electrophysiologic signals for the at least partially undifferentiated cells at the implantation site of the nervous system; and
positioning a second electrode at least proximate to the implantation site of the nervous system, and wherein applying an electrical potential includes placing a subthreshold voltage between the first electrode and the second electrode, wherein the subthreshold voltage is from about 20% to about 50% less than the threshold for generating electrophysiologic signals.

18. The method of claim 1 wherein the at least one electrode includes a first electrode, and wherein the method further comprises:
ascertaining a threshold for eliciting a neural function; and
positioning a second electrode at least proximate to the implantation site of the nervous system, and wherein applying an electrical potential includes placing a subthreshold voltage between the first electrode and the second electrode, wherein the subthreshold voltage is less than the threshold for eliciting the neural function.

19. The method of claim 1 wherein the at least one electrode includes a first electrode, and wherein the method further comprises:
ascertaining a threshold for eliciting a neural function; and
positioning a second electrode at least proximate to the implantation site of the nervous system, and wherein applying an electrical potential includes placing a subthreshold voltage between the first electrode and the second electrode, wherein the subthreshold voltage is from about 30% to about 60% less than the threshold for eliciting the neural function.

20. A method of cell therapy, comprising:
identifying an implantation site of a nervous system of a patient by generating remotely from a location in the nervous system an intended neural activity and determining the site of the nervous system where the generated neural activity is present;
preparing at least partially undifferentiated cells for implantation;
at the implantation site, implanting the at least partially undifferentiated cells directly into tissue of the patient without an implanted substrate;
positioning a first electrode and a second electrode at least proximate to the implantation site of the nervous system, wherein the first electrode and the second electrode are spaced apart from the at least partially undifferentiated cells; and
differentiating the at least partially undifferentiated cells into cells with increased neural characteristics when compared to the at least partially undifferentiated cells by applying an electrical potential between the first electrode and the second electrode and directing an electrical current through the tissue adjacent to the at least partially undifferentiated cells and to the at least partially undifferentiated cells.

21. The method of claim 20 wherein preparing the at least partially undifferentiated cells includes applying an electrical stimulation to the at least partially undifferentiated cells while the at least partially undifferentiated cells are external to a patient.

22. The method of claim 20 wherein the at least partially undifferentiated cells are selected to include stem cells, precursor cells, and/or progenitor cells.

23. The method of claim 20 wherein implanting the at least partially undifferentiated cells directly into tissue without a substrate includes implanting the at least partially undifferentiated cells at least proximate to a spinal cord of the patient.

24. The method of claim 20 wherein implanting the at least partially undifferentiated cells directly into tissue without a substrate includes implanting the at least partially undifferentiated cells at least proximate to a brain of the patient.

25. The method of claim 20 wherein implanting the at least partially undifferentiated cells directly into tissue without a substrate includes implanting the at least partially undifferentiated cells at least proximate to a peripheral nerve of the patient.

26. The method of claim 20 wherein differentiating the at least partially undifferentiated cells into cells with increased neural characteristics includes directing an electrical current between the first electrode and the second electrode at a first voltage until the at least partially undifferentiated cells develop increased action potentials and then applying an electrical current between the first electrode and the second electrode at a second voltage less than the first voltage after the at least partially undifferentiated cells develop increased action potentials.

27. The method of claim 20 wherein differentiating the at least partially undifferentiated cells into cells with increased neural characteristics includes ceasing to apply an electrical current between the first electrode and the second electrode after the at least partially undifferentiated cells develop increased action potentials.

28. The method of claim 20, further comprising ascertaining a threshold for generating action potentials for the at least partially undifferentiated cells at the implantation site of the nervous system, and wherein applying an electrical potential includes applying a subthreshold voltage less than the threshold for generating action potentials.

29. A method of cell therapy, comprising:
preparing fully differentiated neural cells for implantation;
at an implantation site of a nervous system of a patient, implanting the fully differentiated neural cells directly into tissue without an implanted substrate;
positioning at least one electrode at least proximate to the implantation site of the nervous system of the patient;
applying an electrical potential to the at least one electrode while the electrode is at least proximate to the implantation site of the nervous system; and
enhancing connections between native cells and the fully differentiated neural cells by directing an electrical current from the at least one electrode through the tissue surrounding the fully differentiated neural cells.

30. The method of claim 29 wherein preparing fully differentiated neural cells includes applying an electrical stimulation to the fully differentiated neural cells while the fully differentiated neural cells are external to a patient.

31. The method of claim 29, further comprising directing growth of the fully differentiated neural cells by applying an electrical potential to the at least one electrode while the electrode is at least proximate to the implantation site of the nervous system.

32. The method of claim 29, further comprising generating connectivity of the fully differentiated neural cells by applying an electrical potential to the at least one electrode while the electrode is at least proximate to the implantation site of the nervous system.

33. The method of claim 29 wherein implanting the fully differentiated neural cells at an implantation site of a nervous system of a patient includes implanting the fully differentiated neural cells at an implantation site of a spinal cord of the patient.

34. The method of claim 29 wherein implanting the fully differentiated neural cells at an implantation site of a nervous system of a patient includes implanting the fully differentiated neural cells at an implantation site of a brain of the patient.

35. The method of claim 29 wherein implanting the fully differentiated neural cells at an implantation site of a nervous system includes implanting the fully differentiated neural cells at an implantation site of a peripheral nerve of the patient.

36. The method of claim 29 wherein the at least one electrode includes a first electrode, and wherein the method further comprises positioning a second electrode at least proximate to the implantation site of the nervous system of the patient, and wherein applying an electrical potential includes applying a voltage of from about ±1 mV to about ±10 V between the first electrode and the second electrode while the electrodes are at least proximate to the implantation site of the nervous system.

37. The method of claim 29 wherein applying an electrical potential includes generating electrical pulses at a rate of from about 1 to about 1000 Hz.

38. The method of claim 29, further comprising ascertaining a threshold for generating action potentials for the fully differentiated neural cells at the implantation site of the nervous system, and wherein applying an electrical potential includes placing a subthreshold voltage less than the threshold for generating action potentials.

39. The method of claim 29 wherein the at least one electrode includes a first electrode, and wherein the method further comprises:
ascertaining a threshold for generating action potentials for the neural cells at the implantation site of the nervous system; and
positioning a second electrode at least proximate to the implantation site of the nervous system and wherein applying an electrical potential includes placing a subthreshold voltage between the first electrode and the second electrode, wherein the subthreshold voltage is approximately 10-50% less than the threshold for generating action potentials.

40. The method of claim 29 wherein the at least one electrode includes a first electrode, and wherein the method further comprises:
ascertaining a threshold for generating electrophysiologic signals associated with a neural function; and
positioning a second electrode at least proximate to the implantation site of the nervous system and wherein applying an electrical potential includes placing a subthreshold voltage between the first electrode and the second electrode, wherein the subthreshold voltage is less than the threshold for generating electrophysiologic signals.

41. The method of claim 29 wherein the at least one electrode includes a first electrode, and wherein the method further comprises:
ascertaining a threshold for generating electrophysiologic signals for the fully differentiated neural cells at the implantation site of the nervous system; and
positioning a second electrode at least proximate to the implantation site of the nervous system and wherein applying an electrical potential includes placing a subthreshold voltage between the first electrode and the second electrode, wherein the subthreshold voltage is 20-50% less than the threshold for generating electrophysiologic signals.

42. The method of claim 29 wherein the at least one electrode includes a first electrode, and wherein the method further comprises:
ascertaining a threshold for eliciting a neural function; and
positioning a second electrode at least proximate to the implantation site of the nervous system and wherein applying an electrical potential includes placing a subthreshold voltage between the first electrode and the second electrode, wherein the subthreshold voltage is less than the threshold for eliciting the neural function.

43. The method of claim 29 wherein the at least one electrode includes a first electrode, and wherein the method further comprises:
ascertaining a threshold for eliciting a neural function; and
positioning a second electrode at least proximate to the implantation site of the nervous system and wherein applying an electrical potential includes placing a subthreshold voltage between the first electrode and the second electrode, wherein the subthreshold voltage is 30-60% less than the threshold for eliciting the neural function.

44. A method of cell therapy, comprising:
preparing fully differentiated neural cells for implantation;
at an implantation site of a nervous system of a patient, implanting the fully differentiated neural cells directly into tissue without an implanted substrate;
positioning at least one electrode at least proximate to the implantation site of the nervous system of the patient;
applying an electrical potential to the at least one electrode while the electrode is at least proximate to the implantation site of the nervous system; and
directing growth of the fully differentiated neural cells by directing an electrical current from the at least one electrode through the tissue surrounding the fully differentiated neural cells.

45. The method of claim 44 wherein preparing fully differentiated neural cells includes applying an electrical stimulation to the fully differentiated neural cells while the fully differentiated neural cells are external to the patient.

46. The method of claim 44, further comprising generating connectivity of the fully differentiated neural cells by applying an electrical potential to the at least one electrode while the electrode is at least proximate to the implantation site of the nervous system.

47. The method of claim 44 wherein implanting the fully differentiated neural cells at an implantation site of a nervous system of a patient includes implanting the fully differentiated neural cells at an implantation site of a spinal cord of a patient.

48. The method of claim 44 wherein implanting the fully differentiated neural cells at an implantation site of a nervous system of a patient includes implanting the fully differentiated neural cells at an implantation site of a brain of a patient.

49. The method of claim 44 wherein implanting the fully differentiated neural cells at an implantation site of a nervous system includes implanting the fully differentiated neural cells at an implantation site of a peripheral nerve of a patient.

50. The method of claim 44 wherein the at least one electrode includes a first electrode, and wherein the method further comprises positioning a second electrode at least proximate to the implantation site of the nervous system of the patient, and wherein applying an electrical potential includes applying a voltage of from about ±1 mV to about ±10 V between the first electrode and the second electrode while the electrodes are at least proximate to the implantation site of the nervous system.

51. A method of cell therapy, comprising:
preparing at least partially undifferentiated cells for implantation;
applying an electrical stimulation to the at least partially undifferentiated cells while the at least partially undifferentiated cells are external to a patient;
implanting the at least partially undifferentiated cells directly into tissue at an implantation site of a nervous system of the patient after applying the electrical stimulation and without supporting the at least partially undifferentiated implanted cells with a substrate;
positioning a first electrode and a second electrode at least proximate to the implantation site of the nervous system, wherein the first electrode and the second electrode are spaced apart from the at least partially undifferentiated cells; and
differentiating the at least partially undifferentiated cells into cells with increased neural characteristics by directing an electrical current between the first electrode and the second electrodes through the tissue surrounding the at least partially undifferentiated cells and to the at least partially undifferentiated cells.

52. The method of claim 51 wherein the at least partially undifferentiated cells are selected to include stem cells, precursor cells, and/or progenitor cells.

53. The method of claim 51 wherein implanting the at least partially undifferentiated cells directly into tissue at an implantation site of a nervous system of a patient includes implanting the at least partially undifferentiated cells at least proximate to a spinal cord of a patient.

54. The method of claim 51 wherein implanting the at least partially undifferentiated cells directly into tissue at an implantation site of a nervous system of a patient includes implanting the at least partially undifferentiated cells at least proximate to a brain of a patient.

55. The method of claim 51 wherein implanting the at least partially undifferentiated cells directly into tissue at an implantation site of a nervous system includes implanting the at least partially undifferentiated cells at least proximate to a peripheral nerve of a patient.

56. The method of claim 51 wherein applying an electrical potential includes applying a voltage of from about ±1 mV to about ±10 V between the first electrode and the second electrode while the electrodes are at least proximate to the implantation site of the nervous system.

57. The method of claim 51 wherein applying an electrical potential includes generating electrical pulses at a rate of from about 1 to about 1000 Hz.

58. The method of claim 51 wherein differentiating the at least partially undifferentiated cells into cells with increased neural characteristics includes applying an electrical potential between the first electrode and the second electrode at a first voltage until the at least partially undifferentiated cells develop increased action potentials and then applying an electrical potential between the first electrode and the second electrode at a second voltage less than the first voltage after the at least partially undifferentiated cells develop increased action potentials.

59. A method of cell therapy, comprising:
identifying an implantation site of a nervous system of a patient by generating remotely from a location in the nervous system an intended neural activity and determining the site of the nervous system where the generated neural activity is present;
preparing at least partially undifferentiated cells for implantation;
implanting the at least partially undifferentiated cells directly and without an implanted substrate into tissue at the implantation site of the nervous system of the patient;
positioning a first electrode and a second electrode at least proximate to the implantation site of the nervous system, wherein the first electrode and the second electrode are spaced apart from the at least partially undifferentiated cells;
ascertaining a threshold for generating action potentials for the at least partially undifferentiated cells at the implantation site of the nervous system; and
differentiating the at least partially undifferentiated cells into cells with increased action potentials by applying a subthreshold voltage between the first electrode and the second electrode and directing an electrical current through the tissue surrounding the at least partially undifferentiated cells and to the at least partially undifferentiated cells, wherein the subthreshold voltage is approximately 10%-50% less than the threshold for generating action potentials.

60. The method of claim 59 wherein preparing the at least partially undifferentiated cells includes applying an electrical stimulation to the at least partially undifferentiated cells while the at least partially undifferentiated cells are external to the patient.

61. The method of claim 59 wherein the at least partially undifferentiated cells are selected to include stem cells, precursor cells, and/or progenitor cells.

62. The method of claim 59 wherein implanting the at least partially undifferentiated cells directly and without a substrate into tissue at the implantation site of a nervous system of a patient includes implanting the at least partially undifferentiated cells directly into tissue without a substrate at an implantation site of a spinal cord of a patient.

63. The method of claim 59 wherein implanting the at least partially undifferentiated cells directly into tissue without a substrate at the implantation site of a nervous system of a patient includes implanting the at least partially undifferentiated cells directly into tissue without a substrate at an implantation site of a brain of a patient.

64. The method of claim 59 wherein implanting the at least partially undifferentiated cells directly into tissue without a substrate at the implantation site of a nervous system includes implanting the at least partially undifferentiated cells directly into tissue without a substrate at an implantation site of a peripheral nerve of a patient.

65. A method of cell therapy for a location of a brain of a patient, comprising:
identifying a stimulation site by generating remotely from the location in the brain an intended neural activity and determining the site of the brain where the generated neural activity is present;
preparing at least partially undifferentiated cells for implantation;
implanting the at least partially undifferentiated cells at the stimulation site;
positioning at least one electrode at least proximate to the at least partially undifferentiated cells; and
differentiating the at least partially undifferentiated cells into cells with increased neural characteristics when compared to the at least partially undifferentiated cells by applying an electrical potential to the at least one electrode while the electrode is at least proximate to the at least partially undifferentiated cells.

66. The method of claim 65 wherein preparing at least partially undifferentiated cells includes applying an electrical stimulation to the at least partially undifferentiated cells while the at least partially undifferentiated cells are external to a patient.

67. The method of claim 65 wherein the at least partially undifferentiated cells are selected to include stem cells, precursor cells, and/or progenitor cells.

68. The method of claim 65 wherein implanting the at least partially undifferentiated cells at the stimulation site includes implanting the undifferentiated cells directly into tissue without a substrate, and wherein applying an electrical potential includes directing an electrical current from the at least one electrode through the tissue surrounding the at least partially undifferentiated cells and to the at least partially undifferentiated cells.

69. The method of claim 65 wherein the at least one electrode includes a first electrode, and wherein the method further comprises:
positioning a second electrode at least proximate to the at least partially undifferentiated cells; and
resiliently biasing the first electrode and the second electrode against a surface of the brain.

70. The method of claim 65 wherein the at least one electrode includes a first electrode, and wherein the method further comprises:
positioning a second electrode at least proximate to the at least partially undifferentiated cells; and
resiliently biasing at least one of the first electrode and second electrode against a pia mater of the brain.

71. The method of claim 65, further comprising implanting a stimulation apparatus having an integrated pulse system directly coupled to the at least one electrode so that the stimulation apparatus is adjacent to and/or within the skull of the patient, and wherein positioning at least one electrode includes placing the at least one electrode at least proximate to a pia mater.

72. The method of claim 65, further comprising implanting a stimulation apparatus having an integrated pulse system directly coupled to the first electrode so that the stimulation apparatus is adjacent to and/or within the skull of the patient, and wherein positioning at least one electrode includes inserting the at least one electrode into a cortex of the brain.

73. A method of cell therapy, comprising:
identifying an implantation site in a nervous system of a patient where neural activity has changed in response to a change in neural function;
applying an electrical stimulation to cells capable of differentiating into neurons while the cells capable of differentiating into neurons are external to the patient; and
implanting the cells capable of differentiating into neurons at the implantation site after applying the electrical stimulation.

74. The method of claim 73, further comprising applying an electrical stimulation at least proximate to the implantation site after implanting the cells capable of differentiating into neurons at the implantation site.

75. The method of claim 73 wherein identifying an implantation site in a nervous system of a patient where neural activity has changed in response to a change in neural function includes identifying an implantation site in a spinal cord of a patient where neural activity has changed in response to a change in neural function

76. The method of claim 73 wherein identifying an implantation site in a nervous system of a patient where neural activity has changed in response to a change in neural function includes identifying an implantation site in a brain of a patient where neural activity has changed in response to a change in neural function

77. The method of claim 73 wherein identifying an implantation site in a nervous system of a patient where neural activity has changed in response to a change in neural function includes identifying an implantation site in a peripheral nerve of a patient where neural activity has changed in response to a change in neural function

78. The method of claim 73 wherein cells capable of differentiating into neurons are selected from a group consisting of stem cells, precursor cells, or progenitor cells.

79. The method of claim 73 wherein applying an electrical stimulation comprises placing a voltage of ±1 mV to ±10 V across the cells.

80. The method of claim 73 wherein applying an electrical stimulation includes generating electrical pulses at a rate of from about 1 to about 1000 Hz.

81. The method of claim 73, further comprising ascertaining a threshold for generating action potentials for cells capable of differentiating into neurons, and wherein applying an electrical stimulation includes placing a subthreshold voltage across the cells less than the threshold for generating action potentials.

82. The method of claim 73, further comprising ascertaining a threshold for generating action potentials for cells capable of differentiating into neurons, and wherein applying an electrical stimulation includes placing a subthreshold voltage across the cells approximately 10% to approximately 50% less than the threshold for generating action potential.

83. The method of claim 73, further comprising ascertaining a threshold for generating electrophysiologic signals associated with the neural function, and wherein applying an electrical stimulation includes placing a subthreshold voltage across the cells less than the threshold for generating electrophysiologic signals.

84. The method of claim 73, further comprising ascertaining a threshold for generating electrophysiologic signals for cells capable of differentiating into neurons, and wherein applying an electrical stimulation comprises placing a subthreshold voltage across the cells from about 20% to about 50% less than the threshold for generating electrophysiologic signals.

85. The method of claim 73, further comprising ascertaining a threshold for eliciting the neural function, and wherein applying an electrical stimulation includes placing a subthreshold voltage across the cells less than the threshold for eliciting the neural function.

86. The method of claim 73, further comprising ascertaining a threshold for eliciting the neural function, and wherein applying an electrical stimulation includes placing a subthreshold voltage across the, cells from about 30% to about 60% less than the threshold for eliciting the neural function.

87. A method of cell replacement therapy using an apparatus having a support member, a pulse system carried by the support member, and an electrode assembly including first and second electrodes at an interior surface of the support member, comprising:
implanting the support member proximate into a skull of a patient to position the pulse system proximate to the skull and to position the first and second electrodes proximate to a stimulation site on and/or in a brain of the patient;
implanting at least partially undifferentiated cells at the stimulation site;, and
differentiating the at least partially undifferentiated cells into cells with increased neural characteristics when compared to the at least partially undifferentiated cells by controlling the pulse system to apply an electrical potential between the first and second electrodes at least proximate to the stimulation site.

88. The method of claim 87 wherein the at least partially undifferentiated cells can include stem cells, precursor cells, and/or progenitor cells.

89. The method of claim 85 wherein implanting the at least partially undifferentiated cells at the stimulation site includes implanting the at least partially undifferentiated cells directly into tissue without a substrate at the stimulation site, and wherein applying an electrical potential includes directing an electrical current from the electrode assembly through the tissue surrounding the at least partially undifferentiated cells.

90. The method of claim 87, wherein implanting the support member proximate to a skull of a patient includes positioning the first and second electrodes at least proximate to the stimulation site and resiliently biasing at least one of the first and second electrodes against a surface of the brain.

91. The method of claim 87 wherein implanting the support member proximate to a skull of a patient includes positioning the first and second electrodes at least proximate to the stimulation site and resiliently biasing at least one of the first and second electrodes against a pia mater of the brain.

92. A system for cell therapy for a region of a brain of a patient, comprising:
at least partially undifferentiated cells capable of differentiating into cells with increased neural characteristics when compared to the at least partially undifferentiated cells, the at least partially undifferentiated cells being in a state suitable for implantation into the region of the brain;
an implantable support member configured to be implanted into the patient at least partially within a skull of the patient;
a pulse system carried by the support member;
a first electrode carried by the support member, the first electrode being coupled to the pulse system; and
a second electrode carried by the support member, the second electrode being spaced apart from the first electrode, and the second electrode being coupled to the pulse system.

93. The system of claim 92 wherein the at least partially undifferentiated cells capable of differentiating into neurons are selected to include stem cells, precursor cells and/or progenitor cells.

94. The system of claim 92 wherein the support member includes an attachment element and a housing carried by the attachment element, the attachment element being attachable to the skull, and the housing carrying the first and second electrodes.
